(19) United States
(12) Patent Application Publication
(10) Pub. No.: US 2025/0066480 A1
(43) Pub. Date: Feb. 27, 2025

(54) METHODS TO ENHANCE EFFICACY OF COMBINED TARGETING OF IMMUNE CHECKPOINT AND MAPK PATHWAYS

(71) Applicant: THE REGENTS OF THE UNIVERSITY OF CALIFORNIA, OAKLAND, CA (US)

(72) Inventor: Roger S. Lo, LOS ANGELES, CA (US)

(73) Assignee: THE REGENTS OF THE UNIVERSITY OF CALIFORNIA, OAKLAND, CA (US)

(21) Appl. No.: 18/294,767

(22) PCT Filed: Aug. 1, 2022
(86) PCT No.: PCT/US22/74383

§ 371 Date: Feb. 2, 2024

Related U.S. Application Data
(60) Provisional application No. 63/203,862, filed on Aug. 2, 2021.

(51) Int. Cl. C07K 16/28; A61K 38/17; A61K 39/00; A61P 35/04

(52) U.S. Cl. CPC C07K 16/2827; A61K 38/1774; A61P 35/04; C07K 16/2818; A61K 2039/505; A61K 2039/545

(57) ABSTRACT

A method of enhancing efficacy of mitogen-activated protein kinase inhibitor (MAPKi) therapy or immune checkpoint therapy (ICT), as well as a method of suppressing melanoma brain metastasis in a subject, and a method of inhibiting intratumoral M2-like tumor associated macrophages (TAMs) or regulatory T cells (T_{REG}) in a subject comprises (a) pretreating a subject appropriate for and in need of MAPKi therapy by administering one or more doses of ICT to the subject; and (b) subsequent to the pretreating of (a), administering to the subject a combination of MAPKi and ICT. The ICT typically comprises an anti-PD-1 or anti-PD-L1 agent. Optionally, the ICT further comprises additional agents (such as anti-CTLA4 and/or anti-LAG3) to enhance the efficacy of MAPKi therapy.

This application claims benefit of U.S. provisional patent application No. 63/203,862, filed Aug. 2, 2021, the entire contents of which are incorporated by reference into this application.

### STATEMENT REGARDING FEDERALLY SPONSORED RESEARCH

This invention was made with government support under Grant Numbers CA176111, CA215910, CA255837 and CA 168585, awarded by the National Institutes of Health. The government has certain rights in the invention.

### BACKGROUND

Programmed cell death protein-1/programmed death-ligand 1 (PD-1/PD-L1 or PD-1/L1)- and mitogen-activated protein kinase (MAPK)-targeted therapies have revolutionized the treatment of BRAF^{V600MUT} melanoma and beyond. For MAPK inhibitors (MAPKi) consisting of BRAF inhibitor (BRAFi)+MEK inhibitor (MEKi), 5-year survival is under 30%, and acquired resistance occurs within one year in the majority of patients with BRAF^{V600MUT} melanoma. In contrast, for patients with BRAF^{V600WT} melanoma, BRAFi is contraindicated, and MEKi monotherapy provides limited benefits due to innate or rapid development of resistance. Immune checkpoint therapy (ICT) with anti-PD-1 agents results in 30-40% response rates in patients with either BRAF^{V600MUT} or BRAF^{V600WT} melanoma. Combination ICT with anti-PD-1+anti-CTLA-4 agents reduces the rate of innate resistance from 60-70% to 40-50%. Simultaneous initiation of treatments with anti-PD-1/L1 and BRAFi+MEKi (aka triplet therapy) in BRAF^{V600MUT} has been tested in clinical trials (Ascierto et al., 2019; Gutzmer et al., 2020; Ribas et al., 2019) and has been hypothesized to reduce both innate anti-PD-1/L1 resistance and acquired MAPKi resistance.

Retrospective analyses of clinical data suggest that progression on MAPKi is associated with inferior responses to subsequent anti-PD-1 therapy and that any second-line therapy results in inferior outcomes versus the same therapy in the first-line setting (Ackerman et al., 2014; Johnson et al., 2017; Mason et al., 2020; Reijers et al., 2020; Simeone et al., 2017; Tetu et al., 2018). Whether treatment first with ICT until progression alters subsequent MAPKi responsiveness is still unclear. Prospectively, the optimal sequencing of MAPKi vs. ICT is being tested in multiple clinical trials. These trials test one therapy modality until disease progression before switching to the alternative. However, treatment until progression may induce cross-resistance.

Thus, although methods to target the mitogen-activated protein kinase (MAPK) pathway and immune checkpoints (PD-1, PD-L1, CTLA-4) are clinically approved, therapeutic resistance is commonplace. Moreover, resistance of brain metastasis to these therapeutic agents limits patient survival.

There remains a need for methods to improve the efficacy of combined targeting of immune checkpoint and MAPK pathways.

### SUMMARY

The methods described herein meet these needs and others by providing a method of enhancing efficacy of mitogen-activated protein kinase inhibitor (MAPKi) therapy or immune checkpoint therapy (ICT), as well as a method of suppressing melanoma metastasis in a subject, and a method of inhibiting intratumoral M2-like tumor associated macrophages (TAMs) and regulatory or suppressor T (T_{REG}) cells in a subject.

In some embodiments, the method comprises (a) pretreating a subject appropriate for and in need of MAPKi therapy by administering one to two doses of ICT to the subject; and (b) subsequent to the pretreating of (a), administering to the subject a combination of MAPKi and ICT. In some embodiments, the ICT comprises an anti-PD-1 or anti-PD-L1 agent. Optionally, the pretreating with ICT further comprises administering an anti-CTLA-4 and/or anti-LAG3 agent. In some embodiments, the pretreating further comprises administering a modulator of T_{REG} cells, such as an anti-CD20 antibody, or a modulator of M2-like TAMS, such as an agonist of CD206 (e.g., RP832c).

In some embodiments, the subject has a BRAF V600 mutant cancer, and the MAPKi comprises a BRAF inhibitor (BRAFi) plus a MEK inhibitor (MEKi). In some embodiments, the subject has a NRAS, KRAS, and/or NF1 mutant cancer and the MAPKi is a MEKi, ERKi, and/or a type II RAFi (aka pan-RAFi, omni-RAFi).

In some embodiments, the pretreating of (a) consists of administering one to two doses of a single-agent or double-agent ICT over one to eight weeks. In some embodiments, the pretreating of (a) consists of administering one to two doses of a single-agent or double-agent ICT over one to five weeks. The dose to be administered is tailored to the individual ICT and individual patient, taking into account disease progression, patient history, and other indicators of sensitivity or toxicities, as is understood by those skilled in the art. While pretreating typically comprises administering one to two doses of ICT (and, optionally, an anti-CTLA-4 or anti-LAG3 agent, anti-M2 TAM agent, and/or a modulator of T_{REG} cells), it is understood that additional doses during the pretreatment stage are contemplated. For example, a pretreatment dose can be administered every one to four weeks, over the course of one to two months. In some embodiments, a total of four pretreatment doses are administered prior to initiation of MAPKi co-treatment.

In some embodiments, the administering of (b) begins two to five, or up to eight weeks after the initiation of the pretreating of (a). In some embodiments, the administering of (b) begins at least two weeks after the initiation of the pretreating of (a). In some embodiments, the administering of (b) begins at no later than four weeks after the pretreating of (a). In some embodiments, the administering of (b) begins at no later than five weeks after the pretreating of (a). In some embodiments, the administering of (b) begins at no later than eight weeks after the pretreating of (a).

For the method of suppressing melanoma metastasis in a subject, the pretreating of a subject in need of treatment for melanoma comprises administering one to one to two doses of ICT to the subject. In some embodiments, the melanoma metastasis is a brain metastasis. In some embodiments, the melanoma metastasis is an extracranial metastasis. The method can be further enhanced by combining the ICT pretreatment with administration of an anti-CTLA-4 agent and/or a modulator of T_{REG} or M2-like TAMs cells, as mentioned above. The method is of benefit to subjects at risk for brain metastases as well as those at risk of extracranial metastases. Thus, a subject in need of treatment for melanoma brain metastases includes subjects at risk of brain or brain plus extracranial metastases. Subsequent to the pretreating step, the method further comprises administering to the subject a combination of MAPKi and ICT. Since therapeutic resistance of melanoma brain metastasis (MBM) limits patient survival, the sequencing of anti-PD-1/L1 (±anti-CTLA-4 and/or modulator of M2-like TAM or T_{REG} cells) pretreatment before MAPKi combination suppresses MBM and improves survival with robust T-cell clonal expansion in both intracranial and extracranial metastatic sites.

In some embodiments, the method is a method of enhancing the inhibition of M2-like TAMs. In some embodiments, the pretreating further comprises administering a pharmacologic M2-like TAM inhibitor to the subject, in addition to the ICT. The inhibition of M2-like TAMs effects a selective upregulation of pro-inflammatory TAMs in the subject. The inhibition of M2-like TAMs effects a selective upregulation M1-like TAMs.

In some embodiments, the pretreatment comprises administering a CD25-T_{REG}-depleting antibody.

### BRIEF DESCRIPTION OF THE DRAWINGS

FIG. 1 shows progression-free survival of patients with BRAF^{V600MUT} melanoma treated with dabrafenib plus trametinib and stratified by prior exposure to ICT. Results from the S1320 SWOG trial and analyzed by the univariate Cox regression model. ICT, immune checkpoint therapy.

FIGS. 2A-2K demonstrate that two doses of anti-PD-1/L1 before MAPKi combination forestall therapy resistance and induce pro-inflammatory TAM polarization. (2A) Schematic of timelines for subcutaneous tumor progression and for dosing anti-PD-L1 and/or MEK therapies in (2B to 2I) and for tumor sampling in (J and K). Anti-PD-L1 (200 mg/mouse) twice per week IP; MEKi (dosage variable depending on the tumor model) daily PO via chow. Circles indicate regimen and time points for CyTOF analysis in FIG. 2J and scRNA-seq+scTCR-seq analysis in FIG. 3. (2B to 2H) Tumor volumes of YUMM1.7ER (2B), NIL (2C), NILER1-4 (2D), mSK-Mel254 (2E, 2H), CT26 (2F), and KPC (2G) treated with indicated regimens in (2A). Trametinib at 1 (2B, 2C, 2D), 2 (2G), 3 (2E) or 5 (2F) mg/kg/d PO via chow. Anti-PD-L1 (200 mg/mouse) and anti-PD-1 (300 mg/mouse first week only and then 200 mg/mouse) twice per week IP. N=8 tumors/group. Data are means±SEMs (P-values, Student's t test) and representative of two independent experiments. (2I) Overall survival (cutoff tumor volume ≥1000 mm³) of mice bearing YUMM1.7ER tumors treated as indicated. PLX4032, 50 mg/kg/d PO; trametinib, 0.3 mg/kg/d PO. CTRL, historical control. All p-values (logrank test) are for pairwise comparisons relative to mice treated with anti-PD-L1 (d0 to d7) followed by the triplet (anti-PD-L1+PLX4032+trametinib) combination. (2J) t-SNE maps (left) of tumor-infiltrating CD45⁺ cells analyzed by CyTOF in three indicated syngeneic subcutaneous tumor models at time points and in treatment regimens indicated in (2A). Heatmaps (right) showing the expression values of immune phenotypic protein markers normalized to the maximum mean value across subsets. (2K) Frequencies of iNOS⁺ TAMs in the CD45⁺ population of three syngeneic tumor models at time points and treatment regimens indicated in (2A). Mean±SEMs. Pairwise comparisons were performed in (i) vehicle vs. two doses of anti-PD-L1, (ii) MEKi d7 vs. MEKi d7, anti-PD-L1 d0 to d7, (iii) MEKi d7, anti-PD-L1 d7 vs. MEKi d7, anti-PD-L1 d0. P-value, Student's t test. *p<0.05, **p<0.01 and ***p<0.001. See (2A). A color-coded version of FIGS. 2A-2K is available (Wang Y, et al. Cancer Cell. 2021 Oct. 11; 39(10): 1375-1387).

FIGS. 3A-3N show TAM and T-cell phenotypes associated with response to optimized therapy regimen. (3A) Uniform manifold approximation and projection (UMAP) of tumor-infiltrating CD45⁺ cells (n=53,841) analyzed by scRNA-seq. Dissociated live, CD45⁺ cells were pooled from 4 tumors (mSK-Mel254) per regimen and per time point. Inferred cell types are indicated by clusters as denoted by labels based on distinct colors. (3B) UMAP of tumor-infiltrating Mo/MΦ population (n=28,857) analyzed by scRNA-seq. Clusters with differentially expressed genes are denoted by labels based on distinct colors. (3C) Heatmap showing differentially expressed genes (rows) among different Mo/MΦ subpopulations (columns). Representative genes of each cluster are highlighted (right). (3D) Ratios between the proportions of pro- and anti-inflammatory Mo clusters across distinct regimens and time points. See FIG. 2A. (3E) UMAP of tumor-infiltrating T cells (n=13,803) analyzed by scRNA-seq. Clusters with differentially expressed genes are denoted by labels based on distinct colors. (3F) Heatmap showing differentially expressed genes (rows) among different T-cell clusters (columns) and highlighted genes of each cluster (right). (3G) UMAP in (3E) colored by clonality based on scTCR-seq. (3H) Clonal expansion indices of T-cell subpopulations. P-value, Kruskal-Wallis test. (3I) Developmental transition indices of CD8⁺ T_{C} cells with other CD8⁺ subpopulations. Pairwise comparisons were performed between Ki-67^{hi} CD8⁺ T cells and each of the other CD8⁺ subpopulations with the Wilcoxon test. P-values, ***p<0.001. (3J) Relative expansion ratios between T_{C} vs. T_{REG} (left) and Ki-67^{hi} CD8⁺ vs. T_{REG} (right) across different treatment regimens and time points. See FIG. 2A. (3K) Developmental transition indices between CD8⁺ T_{C} and Ki-67^{hi} CD8⁺ T cells across distinct regimens and time points. See FIG. 2A. (3L) Fold changes of top 5 (top) or 10 (bottom) clone sizes for CD8⁺ T_{C} or Ki-67^{hi} CD8⁺ T cells vs. CD4⁺ T_{REG} cells. See FIG. 2A. (3M) Violin plots of Ifng expression in distinct T-cell subpopulations. (3N) Heatmap displaying the scaled mean expression levels of highlighted functional genes (rows) in T_{C} cells across treatment regimens and time points (see FIG. 2A). Gene expression levels were row-scaled for only T_{C} cells. See also FIG. 9 and Table 3. A color-coded version of FIGS. 3A-3N is available (Wang Y, et al. Cancer Cell. 2021 Oct. 11; 39(10): 1375-1387).

FIGS. 4A-4C illustrates functional roles of TAMs and CD8+ T cells in the development of acquired resistance. Tumor volumes of mSK-Mel254 without active treatment or treated with the indicated regimens. Trametinib at 3 mg/kg/d PO; RP-832c (CD206 peptide) at 10 mg/kg/d subcutaneously; anti-PD-L1 (200 mg/mouse IP twice per week); and anti-CD8 antibody at 200 mg/mouse IP initiated one day before any treatment regimen and then twice a week. N=8 tumors/group. Data are means±SEMs (P-values, Student's t test).

FIGS. 5A-5N demonstrate that immune checkpoint blockade before MAPKi co-treatment prolongs MBM suppression and survival. (5A) Schematic of timelines for metastatic tumor progression of Braf^{V600MUT} murine melanoma (YUMM1.7ER) and for dosing anti-PD-L1 and/or MEKi therapies in (B to F). Anti-PD-L1 (200 mg/mouse) IP on d-4 and d-2, when applicable, and, thereafter, twice per week; MEKi, 1 mg/kg/d PO. Circles indicate regimens and time points for TCR-seq analysis in FIG. 6. (5B) Representative ex vivo BLI of organs from necropsies at experimental endpoints (total ventral or dorsal BLI signal in the range of 1e9-1e10, moribund state of health, or death). Scale for radiance, photons/sec. (5C, 5D) Temporal BLI quantification (radiance, photons/sec) based on the dosing timeline in (5A) of dorsal extracranial (5C) or intracranial (5D) tumor burden. Data are mean±SD based on the indicated numbers of mice in the untreated and treatment regimen groups. Pairwise comparisons (mixed model framework with Bonferronic correction for multiple testing) of the group MEKi d0, anti-PD-L1 d-4 vs. the group denoted by the specific corresponding symbol #. #p=0.05-0.001, ##p<0.001. * Indicates death of mouse or mice, resulting in drops in mean BLI values. True or confirmed complete responses or CRs defined in Methods. (5E) In vivo BLI of representative mice from the untreated group vs. all mice treated with MEKi d0 monotherapy at indicated timepoints. All images were adjusted to the same radiance scale. (5F) Survival of untreated mice and mice on each treatment regimen. *p<0.05, **p<0.01, ***p<0.001, ****p<0.0001; ns, not significant (logrank test). Survival criteria indicated in (5B). (5G, 5H) Survival of untreated mice and mice on indicated treatment regimens. P values, logrank test. Day on which BRAFi+MEKi in (5G) were started, defined as day 0, was the same as in (5A) but pushed back by 2 days (day 10 after intracardiac injection) in (5H). Low tumor burden model, MAPKi started on day 8 after IC injection; high tumor burden model, MAPKi started on day 10 after IC injection. (5I, 5J) As in (5C, 5D). (5K) As in (5F). (5L, 5M) As in (5C, 5D), except anti-CTLA-4 at 200 mg/mouse twice a week IP and #p<0.05, ##p<0.01, ###p<0.001, ####p<0.0001. Pairwise comparisons (mixed model framework with Bonferronic correction for multiple testing) of the group BRAFi+MEKi d0, anti-PD-L1 d-4 vs. the group denoted by the specific color of symbol #. (5N) As in (5F) except anti-CTLA-4 at 200 mg/mouse twice a week IP. (5G to 5N) BRAFi, PLX4032, 50 mg/kg/d PO; MEKi, trametinib, 0.3 mg/kg/d PO. See also FIG. 10.

FIGS. 6A-6C show that anti-PD-L1 lead-in before MAPKi co-treatment augments clonal T-cell expansion. (6A) Tumor cell-involved brain and ovarian tissues were collected from mice at time points and in groups as indicated in FIG. 5A (brain, n=2 mice per group, except the no treatment group; ovary, n=1 mouse per group; two geographically distinct regions of each organ site were sampled for TCR-seq analysis). The total sizes of large TCR clones (≥5%) for the α or β chain in tumor-involved brain or ovarian tissues (red dots, average values). Pairwise comparisons were performed between the group of MEKi d0, anti-PD-L1 d-4 vs. each of the other groups with Student's t-test. P-values, *p<0.05, **p <0.01, ***p<0.001. (6B) The fractions of overlapping TCR clones (α or β chain) between two distinct geographic regions of tumor-involved brain tissues from individual mice. (6C) Total sizes of large clones (≥5%) shared by two distinct geographic regions of tumor-involved brain or ovarian tissues in each mouse. Pairwise comparisons were performed as in (6A).

FIG. 7 provides Kaplan-Meier plots of overall survival from the S1320 trial stratified by prior exposure to ICT, Related to FIG. 1. Results from univariate Cox regression model.

FIGS. 8A-8G show the impacts of treatment regimens containing anti-PD-L1 and/or MEKi on tumor cells, CD45+ cells, CD45+ sub-populations, and CD8+ T-cell sub-populations, Related to FIG. 2. (7A) Immunofluorescence of p-ERK levels in tumors of indicated tumor models, with or without trametinib treatment (3 days). DAPI, nuclear stain. Scale bar, 50 μm. Representative images shown for two of five tumors analyzed per group. (8B) Tumor volumes of mSK-Mel254 treated with indicated regimens and on the timelines in FIG. 2A. Trametinib at 3 mg/kg/d. Anti-PD-L1 (200 μg/mouse) and anti-CTLA-4 (200 μg/mouse), both twice per week IP. N=8 tumors/group. Data are means±SEMs (P-values, Student t-test). (8C, 8D, 8E, 8G) Frequencies of CD45+ population in the total live cells (8C), immune cell types in the CD45+ population (8D), CD4+ Th1-like T-cells in the CD45+ population (8E), and CD8+ subsets in the CD8+ population (8G) of three syngeneic tumor models. Mean±SEMs. Pairwise comparisons were performed in (i) vehicle vs. two doses of anti-PD-L1, (ii) MEKi starting d7 vs. anti-PD-L1 d0-d7, MEKi starting d7, (iii) MEKi+anti-PD-L1 starting d7 vs. anti-PD-L1 d0-d7, anti-PD-L1+MEKi starting d7. P-value, Student's t test. *p<0.05, **p<0.01 and ***p<0.001. See FIG. 2A. (8F) Heatmaps showing the expression values of immune phenotypic protein markers across multiple subsets in tumor-infiltrating CD8+ cells analyzed by CyTOF in three indicated syngeneic SC tumor models. The expression values of each marker were normalized to the maximum mean value across subsets. TCM (central memory): CD62L+CD44+GZMB−; TEM (effector memory): CD62L-CD44+GZMB−; TC (cytotoxic): GZMB+; TTD (terminally differentiated): CD62L-CD44-GZMB−.

FIGS. 9A-9G show coupled scRNA-seq/scTCR-seq analysis of mSK-Mel254 tumors from mice undergoing anti-PD-L1 and/or MEKi treatment regimens, Related to FIG. 3. (9A, 9B) UMAP in FIG. 3A was colored by single-cell expression patterns of indicated cell lineage markers (9A) and TCR (9B). (9C to 9E) Frequencies of immune subpopulations in the CD45+ population (9C), Mo/MΦ subpopulations among CD45+ cells (9D), subpopulations among CD4+ T-cells (9E, top) and CD8+ T-cells (9E, bottom). See FIG. 2A. (9F) Boxplots of Tcf1+ stem-like gene signature scores in CD8+ T-cell subsets. Pairwise comparisons were performed in (i) vehicle vs. anti-PD-L1 d0-d7, (ii) MEKi d7 vs. anti-PD-L1 do-d7, MEKi d7, (iii) anti-PD-L1+MEKi d7 vs. anti-PD-L1 d0-d7, anti-PD-L1+MEKi d7. P-value, Wilcoxon test. *p<0.05, ***p<0.001. See FIG. 2A. (9G) Heatmap displaying the scaled mean expression levels of highlighted genes (rows) in each treatment regimen group and time point (column) for TC, T_{REG}, TH1/TH2, NKT and Tγδ subpopulations. See FIG. 2A. Gene expression levels were row-scaled across CD8+ TC, T_{REG}, TH1/TH2, NKT, Tγδ, and Ki-67hiCD8+ subpopulations.

FIGS. 10A-10J demonstrate that anti-PD-L1 lead-in before MAPKi co-treatment optimizes suppression of extracranial and intracranial metastatic progression and augments T-cell clonal expansion, Related to FIGS. 5 and 6. (10A) In vivo BLI of representative mice bearing metastatic YUMM1.7ER cells from the untreated group on indicated timepoints. All images were adjusted to the same radiance scale. (10B) Ex vivo photo (left) and BLI (right) at necropsy of a mouse from the untreated group on day 23 post-IC injection. All images were adjusted to the same radiance scale. (10C, 10D) Temporal BLI quantification (radiance, photons/sec) based on the dosing timeline in FIG. 5A of ventral extracranial (10C) or intracranial (10D) tumor burden. Data are mean±SD based on the indicated numbers of mice in the untreated and treatment regimen groups. Pairwise comparisons (mixed model framework with Bonferronic correction for multiple testing) of the group MEKi d0, anti-PD-L1 d-4 vs. the group denoted by the specific corresponding symbol #. #p=0.05-0.001, ##p<0.001. * Indicates death of mouse or mice, resulting in drops in mean BLI values. True complete responses or CRs defined in Methods. (10E) Flowchart showing the status of surviving mice from experiment in FIG. 5G as they were monitored and grouped beyond 10 weeks (post MAPKi treatment initiation). Specific mice (number designations in 10F) and their final status at the end of long-term follow-ups are shown. (10F) Schematic of the individual natural histories of long-term surviving mice from experiment in FIG. 5G and their disease, treatment, and vital status. (10G) Tumor cell-involved brain and ovarian tissues were collected from mice at time points and in groups as indicated in FIG. 5A (brain, n=2 mice per group, except the no treatment group; ovary, n=1 mouse per group; two geographically distinct regions of each organ site were sampled for TCR-seq analysis). Dot plot showing the diversity and gini clonality indices for α or β chain in brain or ovarian tissues (red dots, average values). Pairwise comparison was performed between MEKi d0, anti-PD-L1 d-4 and each of the other treatment groups with Student's t test. P-values, *p<0.05, **p<0.01, ***p<0.001. (10H) The total sizes of large TCR clones (≥8%) for the α or β chain in tumor-involved brain or ovarian tissues (hatched dots, average values). Pairwise comparisons were performed as in 10E. (10I) The total sizes of largest, top 5, and top 10 TCR clones (≥8%) for the α or β chain in tumor-involved brain or ovarian tissues (hatched dots, average values). Pairwise comparisons were performed as in 10E. (10J) Venn plots showing the overlapping fractions of TCR clones (α or β chain) between brain and ovarian tissues in each individual.

### DETAILED DESCRIPTION

The invention described herein is based on the surprising discovery that sequencing of just two doses of anti-PD-1/L1 prior to combining with MAPKi maximizes antitumor efficacy. Data presented here support the conclusions that anti-PD-1/L1 therapy primes a more durable MAPKi response and that MAPKi combination therapy overcomes innate anti-PD-1/L1 resistance. These findings provide a basis for methods to enhance the efficacy of MAPKi therapy as well as of ICT, and to suppress melanoma brain metastases. In addition, the efficacy of treatment can be further enhanced by inhibition of M2-like TAMs, and of regulatory T cells. The data described herein demonstrate that the superior antitumor activity of the regimen consisting of anti-PD-1/L1 lead-in followed by MAPKi combination was positively linked to multiple cell types, including M1 TAMs, Th1 CD4+ T-cells, as well as CD8+ cytotoxic and proliferative T cells and their persistence.

### Definitions

All scientific and technical terms used in this application have meanings commonly used in the art unless otherwise specified. As used in this application, the following words or phrases have the meanings specified.

As used herein, “anti-PD-1 therapy” means treatment with an anti-PD-1 antibody (nivolumab/BMS-936558/MDX-1106, pembrolizumab/MK-3475, Pidilizumab), and/or an anti-PD-L1 antibody (BMS-986559, MPDL3280A, and MEDI4736).

As used herein, “combinatorial therapy” means MAPK targeted therapy, anti-CTLA-4 immunotherapy in any combination, with or without anti-PD-1 antibody and/or anti-PD-L1 antibody treatment.

As used herein, “MAPK/ERK kinase (MEK)” refers to a mitogen-activated protein kinase also known as mitogen-activated protein kinase (MAPK) or extracellular signal-regulated kinase (ERK).

MEK, also known as mitogen-activated protein kinase kinase and MAP2K, is a kinase enzyme that phosphorylates mitogen activated protein kinases (MAPKs), ERK, p38 and JNK. Seven MEK subtypes have been identified, all mediate cellular responses to different growth signals.

BRAF (v-raf murine sarcoma viral oncogene homolog B1) is a serine/threonine protein kinase that plays a critical role in the RAS-RAF-MEK-ERK mitogen activated protein kinase (MAPK) cell signalling pathway.

As used herein, “therapy”, “treatment” or “treating” means any administration of a therapeutic agent according to the present disclosure to a subject (e.g. human) having or susceptible to a condition or disease, such as cancer, for the purpose of: preventing or protecting against the disease or condition, that is, causing the clinical symptoms not to develop; inhibiting the disease or condition, that is, arresting or suppressing the development of clinical symptoms; or relieving the disease or condition that is causing the regression of clinical symptoms. In some embodiments, the term “therapy”, “treatment” or “treating” refers to relieving the disease or condition, i.e. which is causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment. The presence of a genetic mutation or the predisposition to having a mutation may not be alterable. However, prophylactic treatment (prevention) as used herein has the potential to avoid/ameliorate the symptoms or clinical consequences of having the disease engendered by such genetic mutation or predisposition. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.” The term “effective amount” refers to that amount of a therapeutic agent that is sufficient to effect treatment when administered to a subject in need of such treatment. The effective amount will vary depending upon the specific activity of the therapeutic agent being used, the severity of the patient's disease state, and the age, physical condition, existence of other disease states, and nutritional status of the patient. Additionally, other medication the patient may be receiving will affect the determination of the effective amount of the therapeutic agent to administer.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA. 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Methods

The methods described herein meet these needs and others by providing a method of enhancing efficacy of mitogen-activated protein kinase inhibitor (MAPKi) therapy or immune checkpoint therapy (ICT), as well as a method of suppressing melanoma metastasis in a subject, and a method of inhibiting M2-like tumor associated macrophages (TAMs) in a subject.

In some embodiments, the method comprises (a) pretreating a subject appropriate for and in need of MAPKi therapy by administering one to two doses of ICT to the subject; and (b) subsequent to the pretreating of (a), administering to the subject a combination of MAPKi and ICT. In some embodiments, the ICT comprises an anti-PD-1 or anti-PD-L1 agent. Optionally, the pretreating with ICT further comprises administering an anti-CTLA-4 agent or an anti-LAG3 agent. In some embodiments, the pretreating further comprises administering a modulator of T_{REG} cells, such as a CD25-T_{REG}-depleting antibody or a modulator of M2-like TAMs, such as an agonist of CD206 (e.g., RP832c).

In some embodiments, the subject has a BRAF V600 mutant cancer, and the MAPKi comprises a BRAF inhibitor (BRAFi) plus a MEK inhibitor (MEKi). In some embodiments, the subject has a NRAS, KRAS, and/or NF1 mutant cancer and the MAPKi is a MEKi, ERKi, and/or a type II RAFi (aka pan-RAFi, omni-RAFi).

In some embodiments, the pretreating of (a) consists of administering one to two doses of a single-agent, double-agent, or triple-agent ICT over one to eight weeks. In some embodiments, the pretreating of (a) consists of administering one to two doses of a single-agent, double-agent, or triple-agent ICT over one to five weeks. The dose to be administered is tailored to the individual ICT and individual patient, taking into account disease progression, patient history, and other indicators of sensitivity or toxicities, as is understood by those skilled in the art. While pretreating typically comprises administering one to two doses of ICT (and, optionally, an anti-CTLA-4/LAG3 agent and/or a modulator of T_{REG} or M2-like TAM cells), it is understood that additional doses during the pretreatment stage are contemplated. For example, a pretreatment dose can be administered every one to four weeks, over the course of one to two months. In some embodiments, a total of four pretreatment doses are administered prior to MAPKi treatment begins.

In some embodiments, the administering of (b) begins two to eight weeks after the initiation of the pretreating of (a). In some embodiments, the administering of (b) begins two to five weeks after the initiation of the pretreating of (a). In some embodiments, the administering of (b) begins at least two weeks after the initiation of the pretreating of (a). In some embodiments, the administering of (b) begins at no later than eight weeks after the pretreating of (a). In some embodiments, the administering of (b) begins at no later than four weeks after the pretreating of (a).

For the method of suppressing melanoma metastasis in a subject, the pretreating of a subject in need of treatment for melanoma comprises administering one to one to two doses of ICT to the subject. In some embodiments, the melanoma metastasis is a brain metastasis. In some embodiments, the melanoma metastasis is an extracranial metastasis. The method can be further enhanced by combining the ICT pretreatment with administration of an anti-CTLA-4/LAG3 agent and/or a modulator of T_{REG} or M2-like TAM cells, as mentioned above. The method is of benefit to subjects at risk for brain metastases as well as those at risk of extracranial metastases. Subsequent to the pretreating step, the method further comprises administering to the subject a combination of MAPKi and ICT. Since therapeutic resistance of melanoma brain metastasis (MBM) limits patient survival, the sequencing of anti-PD-1/L1 (±anti-CTLA-4/LAG3 and/or modulator of T_{REG} or M2-like TAM cells) pretreatment before MAPKi combination suppresses MBM and improves survival with robust T-cell clonal expansion in both intracranial and extracranial metastatic sites.

In some embodiments, the method is a method of enhancing the inhibition of M2-like TAMs. In some embodiments, the pretreating further comprises administering a pharmacologic M2-like TAM inhibitor to the subject, in addition to the ICT. The inhibition of M2-like TAMs effects a selective upregulation of pro-inflammatory TAMs in the subject. The inhibition of M2-like TAMs effects a selective upregulation M1-like TAMs.

In some embodiments, the pretreatment comprises administering a CD25-T_{REG}-depleting antibody.

### Kits & Compositions

The invention provides kits and/or compositions comprising one or more reagents and/or therapeutic agents suitable for use in the methods described herein, and optionally, one or more suitable containers containing reagents and/or agents of the invention. Such kits can comprise a carrier, package or container that is compartmentalized to receive one or more containers such as vials, tubes, and the like, each of the container(s) comprising one of the separate elements to be used in the method. The reagents and/or agents of the kit may be provided in any suitable form, including frozen, lyophilized, or in a pharmaceutically acceptable buffer such as TBS or PBS.

Agents include an inhibitor of MAPKi, an anti-CTLA4 agent, an agonist of CD206, such as RP832c, and/or a modulator of regulatory T cells, such as CD25-T_{REG}-depleting antibody. Examples of CD25-T_{REG}-depleting antibodies include, but are not limited to, daclizumab. Agents can be provided in the form of a composition suitable for administration to a subject in accordance with the methods described here.

The kit of the invention will typically comprise the container(s) described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use, such as those described herein. Directions and or other information can also be included on an insert, which is included with the kit.

### EXAMPLES

The following examples are presented to illustrate the present invention and to assist one of ordinary skill in making and using the same. The examples are not intended in any way to otherwise limit the scope of the invention.

### Example 1: Anti-PD-1/L1 Lead-In Before MAPK Inhibitor Combination Maximizes Antitumor Immunity and Efficacy

This Example demonstrates that rationally sequencing and combining PD-1/L1- and MAPK-targeted therapies can overcome innate and acquired resistance. Since increased clinical benefit of MAPK inhibitors (MAPKi) is associated with prior immune checkpoint therapy, this Example compares the efficacies of sequential and/or combinatorial regimens in subcutaneous murine models of melanoma driven by Braf^{V600}, Nras, or Nf1 mutations as well as colorectal and pancreatic carcinoma driven by Kras^{G12C}. Anti-PD-1/L1 lead-in preceding MAPKi combination optimizes response durability by promoting pro-inflammatory polarization of macrophages and clonal expansion of IFNg^{hi}, CD8⁺ cytotoxic and proliferative (versus CD4⁺ regulatory) T cells that highly express activation genes. Since therapeutic resistance of melanoma brain metastasis (MBM) limits patient survival, this Example demonstrates that sequencing anti-PD-1/L1 therapy before MAPKi combination suppresses MBM and improves mouse survival with robust T-cell clonal expansion in both intracranial and extracranial metastatic sites. These results support brief anti-PD-1/L1 (±anti-CTLA-4) dosing prior to MAPKi co-treatment to suppress therapeutic resistance.

To date, the impact of shorter exposures to one therapy (to generate a priming effect) before switching to or combining with another therapy has not yet been evaluated. This sequential-combinatorial fusion may avoid the development of cross-resistance, raise the threshold for resistance evolution by stacking multiple therapeutic mechanisms of action, and permit one mode of therapy to prime responsiveness to the other, thereby creating synergy. Importantly, prior studies have implicated a role of antitumor immunity in prolonging, clinically and preclinically, the durability of MAPKi responses (Hong et al., 2021; Hugo et al., 2015).

Since prior ICT exposure seems to prime subsequent MEKi responsiveness in patients with NRAS^{MUT} melanoma (Dummer et al., 2017), we test the hypothesis that the same association exists in patients with BRAF^{V600MUT} melanoma. We also test the hypothesis that brief anti-PD-1/L1 dosing or lead-in before MAPKi combination maximizes antitumor efficacy and identify intratumoral immune cell phenotypes that associate with superior efficacy. Since MAPKi or ICT appears to be less durably active against intracranial (vs. extracranial) melanoma metastases (Flaherty et al., 2012; Ribas et al., 2016), we evaluate whether the optimal sequential-combinatorial regimen suppresses resistance in an organ-specific context. In particular, BRAFi+MEKi elicit lower response rates against clinical melanoma brain metastasis (MBM) (Davies et al., 2017), and clinically acquired MAPKi resistance emerges preferentially in the brain (Seifert et al., 2016). Thus, we develop a murine model of experimental melanoma metastases where MBM limits survival and compare the relative efficacies of anti-PD-1/L1 (±anti-CTLA-4) and MAPKi sequential-combinatorial regimens and their impacts on T-cell clonality.

### Experimental Models and Subject Details

### Human Subjects

Patient characteristics and sample sizes are presented in Table 1. Patients were enrolled in the S1320 clinical trial with informed consent obtained from all patients and participation of sites approved by local institutional review boards.

### Mice

C57BL/6 and Balb/c (for subcutaneous models) were obtained from the Radiation Oncology breeding colony at UCLA (Los Angeles, CA). C57BL/6 (for experimental metastasis model) were purchased from Jackson Laboratory. Female mice were used at 6-8 weeks of age. All animal experiments were conducted according to the guidelines approved by the UCLA Animal Research Committee.

### Subcutaneous Syngeneic Tumor Models

For syngeneic subcutaneous tumor models, C57BL/6 (YUMM1.7ER, NIL, NILER1-4, mSK-Mel254 and KP4662) or Balb/c (CT26) mice were injected on both flanks with one million cells. Tumors were measured with a calliper every 2 days, and tumor volumes were calculated using the formula (length×width²)/2. Once tumors reached a size of 120-140 mm³, mice were assigned randomly into experimental groups. Special mouse diets (for C57BL/6 and Balb/c) were generated by incorporating trametinib at 1, 2, 3 or 5 mg/kg to facilitate daily drug dosing and to reduce animal stress (TestDiet). The combination of BRAFi+MEKi (PLX4032 50 mg/kg/day and trametinib 0.3 mg/kg/day; both resuspended in 10% DMSO with 0.1% methylcellulose) was administered to mice via oral gavage (subcutaneous model) or incorporated in chow (TestDiet). Anti-PD-L1 (200 μg/mouse, BioXcell), anti-PD-1 (300 μg/mouse for the first 2 doses, then 200 μg/mouse, Leinco Technologies) and anti-CTLA-4 (200 μg/mouse, BioXcell) were intraperitoneally administered twice per week. Anti-CD8 antibody was intraperitoneally administrated (200 μg/mouse, BioXcell) on day-1, day 0 or on day 6, day 7, and then twice a week. RP-832c was subcutaneously administrated (10 mg/kg, Riptide Bioscience) from day 0 to day 14, daily. On indicated days, tumors were excised from mice, minced, and digested to single-cell suspensions using a tumor dissociation kit and gentleMACS™ Octo Dissociator (Miltenyi Biotech) and prepared for CyTOF staining or/and 10× Genomics single cell RNA sequencing followed by sorting for live CD45⁺ cells.

### Experimental Metastasis of Syngeneic Melanoma

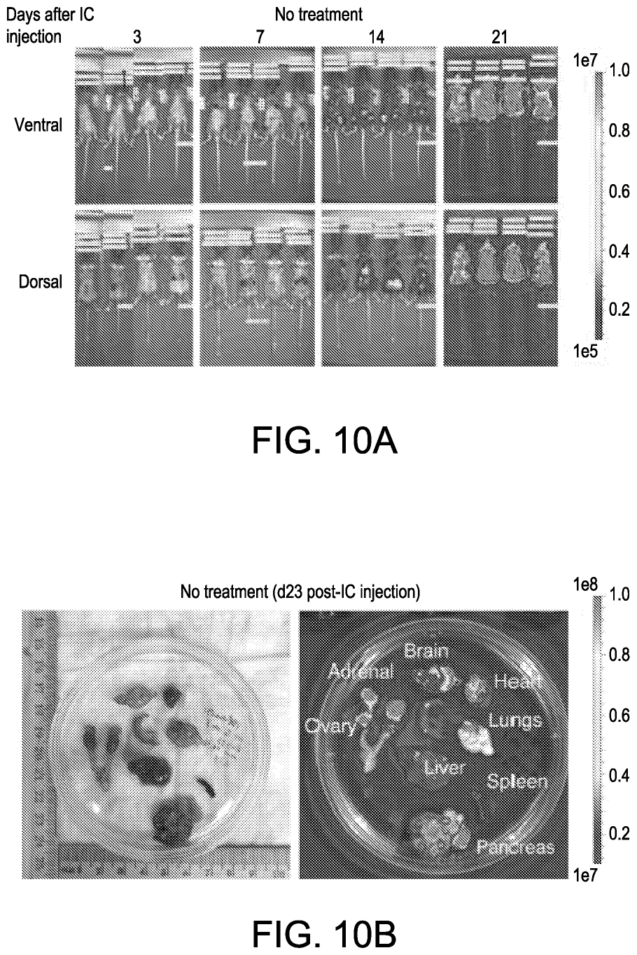

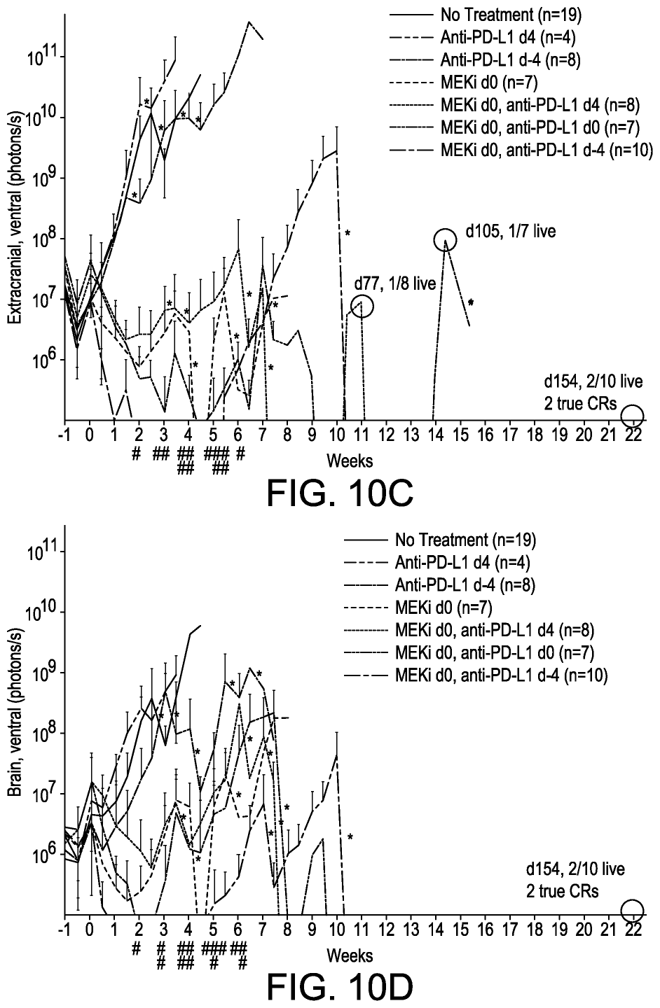

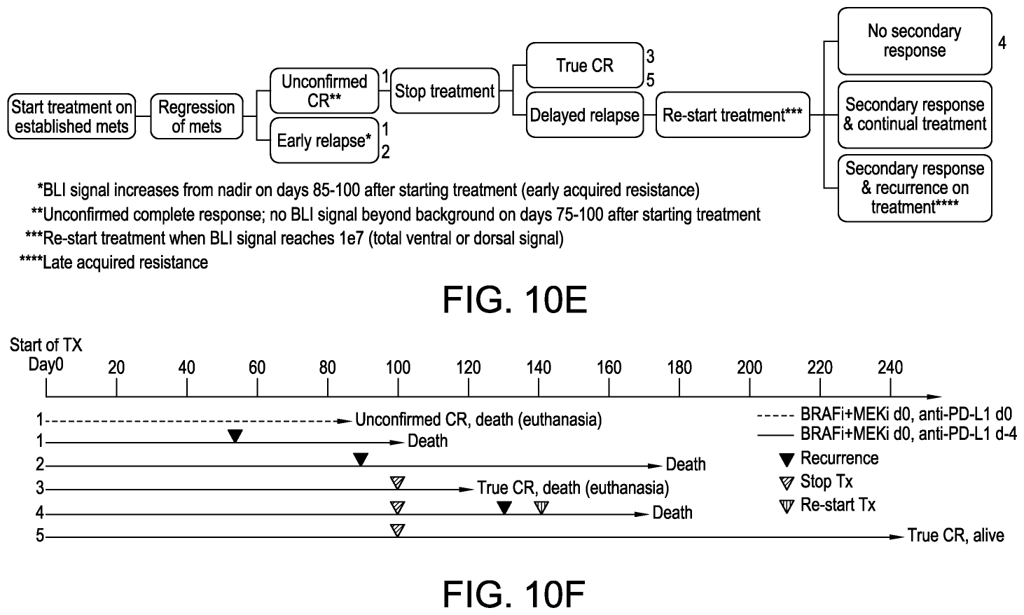

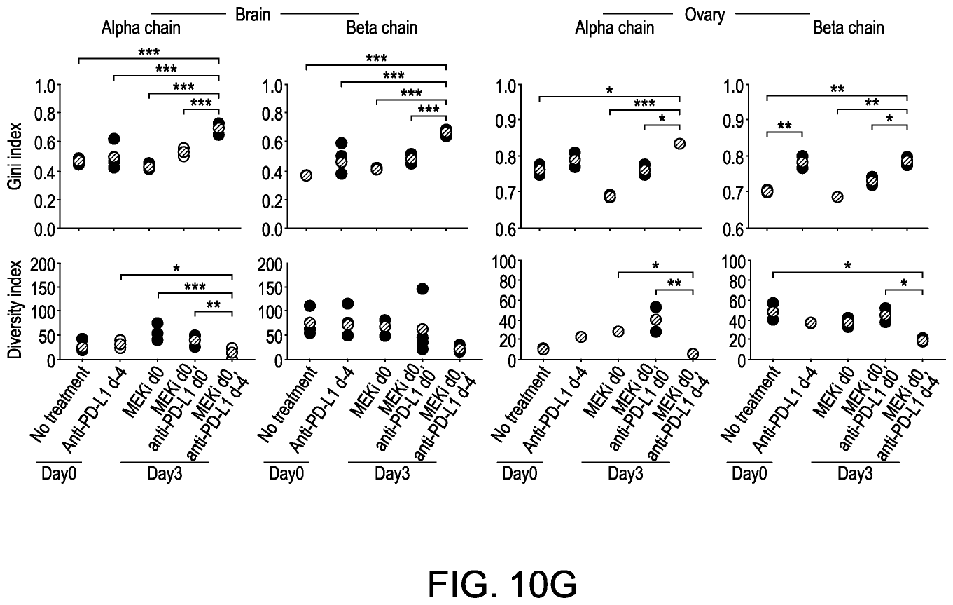

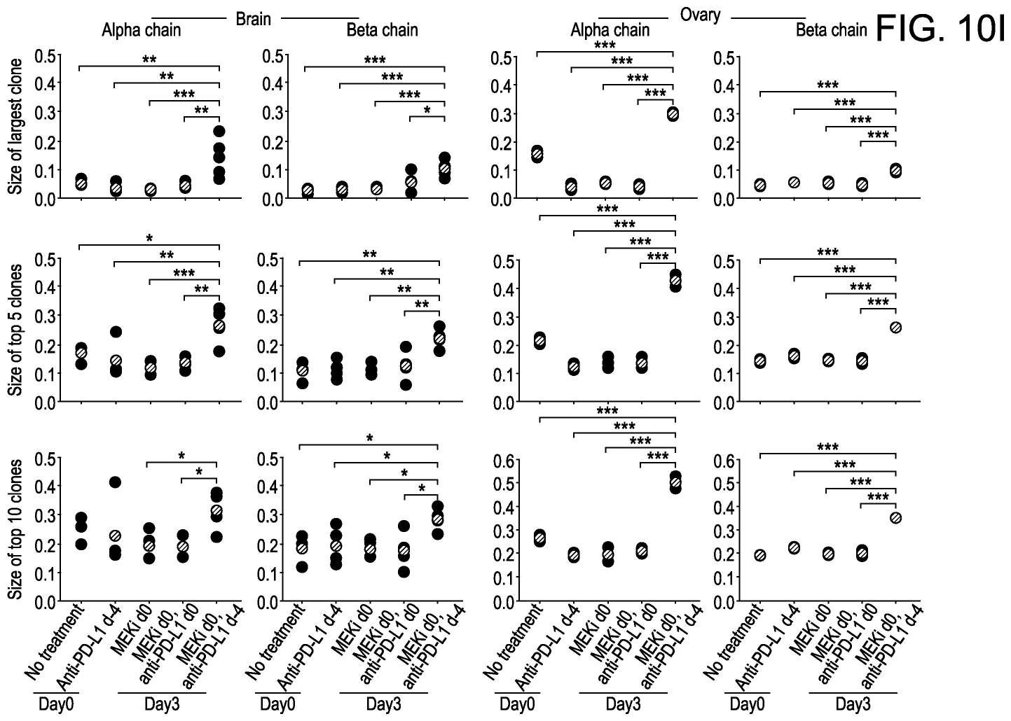

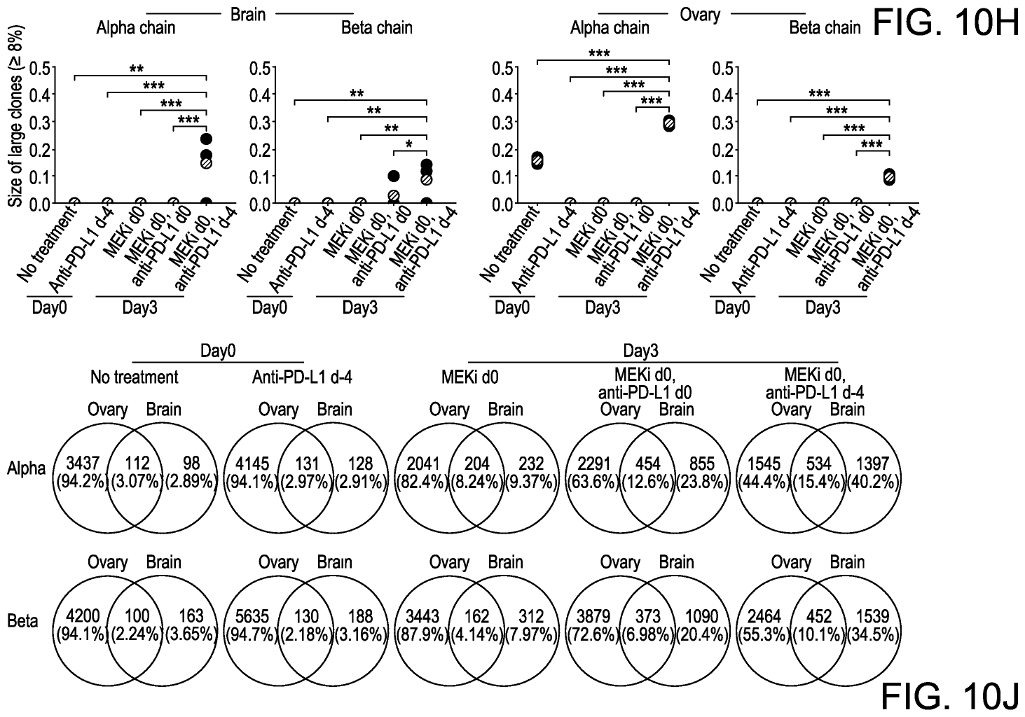

YUMM1.7ER melanoma cells were engineered to express firefly luciferase (YUMM1.7ER-luc). Before every IC injection, cells were selected for stable luciferase expression by blasticidin treatment, yielding cells (1 million) that emitted luciferase light units of 5e8 to 5e9 in vitro. C57BL/6 (6-8 weeks old, female), after anesthesia with vaporized isoflurane (2.0-2.5%), were injected with 1×10⁶ cells/mouse into the left ventricle for intracardiac inoculation. Progression of metastatic tumor burden and treatment effects in mice were monitored in vivo and ex vivo by bioluminescent imaging (BLI). We acquired BLI on both ventral and dorsal sides twice weekly until study endpoints (total ventral or dorsal BLI signal in the range of 1e9-1e10, moribund, or death). Mice were anesthetized with vaporized isoflurane; D-luciferin (150 mg luciferin/kg, Caliper Life Sciences) dosed by intraperitoneal injections; and, 10 minutes after injections, luciferase activities were measured with IVIS Lumina II Imaging System (PerkinElmer). Image analysis was performed with Living Image 4.7.2 Version (PerkinElmer). For ex vivo BLI, mice were euthanized 10 minutes after intraperitoneal injection of D-luciferin by cervical dislocation and organs were excised and imaged ex vivo by the IVIS system. Special mouse diets (for C57BL/6 mice) were generated by incorporating trametinib at 1 mg/kg/day or PLX4032 at 50 mg/kg/day plus trametinib at 0.3 mg/kg/day to facilitate daily drug dosing and to reduce animal stress (TestDiet). Anti-PD-L1 (200 μg/mouse, BioXcell) or anti-PD-1 (200 μg/mouse, Leinco Technologies) was intraperitoneally administered twice per week. Anti-CTLA-4 (200 μg/mouse, BioXcell) was intraperitoneally administered every four days starting at d-4 and ending d+4. We visualized BLI data on a logarithmic scale (means and corresponding standard deviations). Down error bars were not presented when they extended to zero or negative values. For mice in the experimental metastasis model treated with MEKi+anti-PD-L1 regimens, mice with no BLI signal beyond background for >˜60 days after starting treatment were deemed to have unconfirmed complete responses (CRs). For mice in the experimental metastasis model treated with BRAFi+MEKi+anti-PD-1/L1 regimens, mice with no BLI signal beyond background for ˜75-100 days after starting treatment were deemed to have unconfirmed complete responses (CRs). To confirm CRs in these mice, treatment was stopped. Mice were confirmed to display complete CRs if only background radiance was detected twice weekly for two consecutive months after treatment cessation or until the experimental endpoint (death by euthanasia in situations of deteriorating health), whichever comes first. If tumor burden becomes detectable after treatment cessation, the protocol in the flowchart (FIG. 10E) was followed.

### Cell Lines

All mouse cancer cell lines were routinely tested for mycoplasma and profiled and identified by RNA-seq and the GenePrint 10 system (Promega) at periodic intervals during the course of this study for banking and experimental studies. All cell lines were maintained in either DMEM (YUMM1.7ER, YUMMER1.7-luc, NIL, NILER1-4, mSK-Mel254 and KP4662) or RPMI (CT26) supplemented with high glucose with 10% heat-inactivated FBS (Omega Scientific) and 2 mM glutamine in humidified, 5% CO2 incubator. To establish YUMM1.7ER-luc, the pHIV-Luc-Zsgreen vector (Addgene) was subcloned into the lentiviral vector pLV-EF1a-IRES-Blast (Addgene). YUMM1.7ER cells transduced by luciferase-expressing lentiviruses were selected using blasticidin (Sigma-Aldrich).

### Method Details

### Mass Cytometry of Murine Tumors

2×10⁶ or fewer cells were incubated with 2% of FBS in PBS with 25 μg/ml of 2.4G2 antibody at 4° C. for 10 min prior to surface staining with an antibody cocktail at 4° C. for 30 min in a 50 μL volume. Cells were incubated with 2.5 μM 194 Pt monoisotopic cisplatin (Fluidigm) at 4° C. for 1 min. Cells were then washed twice with FACS buffer and barcoded using palladium metal barcoding reagents according to manufacturer's protocol (Fluidigm). Subsequently, fixation and permeabilization were performed using the Foxp3 fix and permeabilization kit according to the manufacturer's protocol (Fluidigm). Cells were then stained with an intracellular stain antibody cocktail (Foxp3, Ki67, granzyme B, T-bet, iNos, Eomes) for 30 min at room temperature. Cells were then washed twice with Foxp3 permeabilization buffer, twice with FACS buffer, and incubated overnight in 1.6% PFA PBS with 100 nM iridium nucleic acid intercalator (Fluidigm). Cells were then washed twice with PBS with 0.5% BSA, filtered, and washed twice with water with 0.1% BSA prior to analysis. Samples were analyzed using a Helios mass cytometer based on the Helios 6.5.358 acquisition software (Fluidigm).

### Single Cell 5′ Gene Expression and V(D)J Sequencing

Four different tumors per regimen and per time point were dissociated to single-cell suspensions using a tumor dissociation kit (Miltenyi Biotech) and gentleMACS™ Octo Dissociator (Miltenyi Biotech). 5×10⁵ cells per tumor were pooled together (2×10⁶ in total) as one sample for each regimen and each time point. Cells were incubated with 20% FBS in PBS with 25 mg/ml of anti-mouse CD16/CD32 antibody (eBioscience) at 4° C. for 10 min to minimize background antibody binding. Then cells were stained with BV510-anti-CD45 (1 mg/mL, Biolegend) and PerCP-anti-TER119 (2 mg/mL, Biolegend) at room temperature for 20 minutes, followed by 7AAD (10 mL in 500 mL PBS per sample, BD Pharmigen) staining for 5 minutes on ice. Cells after staining were sorted by BD FACSAria II sorting system to harvest the BV510 (CD45) positive and PerCP (TER119, 7AAD) negative population as live CD45⁺ cells. Cells recovered were subjected to 10× Genomics standard protocol for coupled scRNA-seq and scTCR-seq library preparation using Chromium Next GEM Single Cell 5′ Library and Gel Bead Kit v1.1 (10× Genomics) and V(D)J Enrichment Kit for Mouse T Cells (10× Genomics). Libraries were sequenced by Novaseq 6000 S2 flow cell with 2×50 reads targeting a minimum of 20,000 read pairs per cell for scRNA-seq library and 5,000 read pairs per cell for scTCR-seq library.

### Generation of Bulk Tumor TCR-Seq Data

Total RNA was extracted from frozen tissue stored in RNALater using the QIAGEN All Prep DNA/RNA Mini Kit. RNA quality was measured using Bioanalyzer (Agilent). 710 ng-1,000 ng of RNA from ovary and brain tissues (RNA Integrity Number (RIN) score >7) was used as input to construct libraries with the QIAGEN QIAseq Immune Repertoire RNA Library Kit—T cell Receptor Panel (Qiagen). Briefly, RNA was reverse transcribed using a pool of TCR gene-specific primers against the constant region for the T cell receptor alpha, beta, gamma, and delta genes. The resulting cDNA was then ligated to an oligo containing one side of sample index and unique molecular index (UMI). After reaction cleanup, a single primer extension was used to capture the T cell receptor using a pool of gene-specific primers. The resulting captured sequences were amplified and purified using QIAseq beads. Libraries were then sample-indexed on the other side by using a unique sample index primer and an universal primer to amplify the library and introduce platform-specific adapter sequences. The dual-indexed sample PCR fragment was purified and then quantified for absolute quantification of amplifiable libraries (DNA with adaptors at both ends) in triplicate by real-time qPCR using QIAGEN QIAseq Library Quant Array Kit. For sequencing, each library was diluted to 4 nM, pooled, and denatured. 12 μM of denatured library pool was run with QIAseq A Read1 Primer on Illumina NextSeq 500 Mid Output Kit using v2.5 chemistry for 300 cycles with an asymmetrical paired end 261/41 bp read for CDR3 region.

### Tissue Staining

For immunofluorescence (IF), tissues were fixed in formalin followed by embedding in paraffin. After deparaffinization and rehydration, tissue sections were subjected to heat for antigen retrieval. After tissue sections were permeabilized and blocked, primary antibodies (phospho-ERK1/2 (Cell Signaling Technology, #4370)) were added overnight. IF was performed with Alexa Fluor-conjugated secondary antibodies (Life Technologies, #A21429). Nuclei were counterstained by DAPI (ThermoFisher Scientific). Signals were captured with a Zeiss microscope (AXIO Imager A1) mounted with a charge-coupled device camera (Retiga EXi QImaging), and the images captured by Image-pro plus 6.0.

A detailed listing of all reagents and resources used in these methods is available at Wang et al., 2021, Cancer Cell 39, 1375-1387. Raw sequencing files of scRNA-seq, scTCR-seq, and bulk TCR-seq data have been deposited at GEO (GSE177902) and are publicly available.

### Quantification and Statistical Analysis

### Clinical Trial Data Analysis

Details of S1320 (ClinicalTrials.gov Identifier: NCT02196181; SWOG Identifier: S1320) have been published with the primary outcome manuscript (Algazi et al., 2020). Association between prior ICT and categorical and quantitative variables were compared using Fisher's exact and Wilcoxon tests, respectively. PFS and OS were estimated using the Kaplan-Meier method, and Cox regression models were used to evaluate associations.

### CyTOF Data Analysis

Mass cytometry flow cytometry standard (FCS) data files were concatenated, bead-normalized and debarcoded using Helios software (Fluidigm), and then exported into individual files for each sample. Total CD45⁺ and CD8⁺ and CD4⁺ T-cell populations were manually identified and exported using negative and positive gating strategies of lineage markers in Cytobank (Kotecha et al., 2010). We applied Cytofkit (Chen et al., 2016) to perform the t-Distribution Stochastic Neighbor Embedding (t-SNE) analysis separately on the manually gated CD45⁺, CD4⁺ and CD8⁺ populations. We selected 5,000 (in sub-clustering CD45⁺) or 2,000 events (in sub-clustering CD4⁺/CD8⁺ T-cells) in each sample to ensure equal representation of cells across samples. All the cell lineage markers in the immune panel were used in CD45⁺ analysis. For T-cell analysis, all markers excluding the following were used: CD90, CD14, F480, Ly6G, CXCR2, CD19, and CD335. We chose the 3,000 iterations, perplexity of 30 and theta of 0.5, as the standard t-SNE parameters. Mean intensity values of markers in each cluster were calculated and visualized via heatmaps. Cells were assigned to different functional populations on the basis of the local gradient expression of known cell lineage markers. The percentages of different immune cell subsets were calculated for each sample.

Analysis of scRNA-Seq Data

Alignment to GRCm38 reference genome, barcode and unique molecular identifier (UMI) counting were performed using Cell Ranger (10× Genomics, v2.1.0). Seurat package (Butler et al., 2018) was used for downstream analysis. Cells with fewer than 500 genes detected or greater than 10% mitochondrial RNA content were excluded from further analysis. Raw UMI counts were normalized to UMI count per million total counts and log-transformed. Variable genes were detected based on average expression and dispersion for each dataset independently. We then use CellCycleScoring function to calculate scores of S and G2/M cell cycle phases for each cell. Single cells from different conditions were integrated into a single assay based on variable genes identified from each sample. We then use the ScaleData function to calculate scaled z-scores of each variable gene in the integrated assay and regress out the effect of number of genes per cell, mitochondrial RNA content, and cell cycle scores (S phase score and G2/M phase score). This scaled data set was then used for principal component analysis (PCA) for cells. Clusters and UMAP projections were generated based on the top 30 PCA dimensions. Clusters were annotated based on expression of known marker genes, including Cd14 (myeloid), Igtam, Csf1r (monocyte/macrophage), Flt3 (dendritic cell), S100a8, S100a9 (neutrophil), Ncr1 (NK cell), Cd19, Cd79a (B-cell), Cd3d, Cd3e, Cd3g (T-cell). Cell clusters co-expressing markers of multiple cell types were defined as doublets and excluded from further analysis. We next isolated the monocyte/macrophage and T-cell populations identified from the broad clustering analysis and performed re-clustering analysis on them separately. Cells were re-clustered as described above and functional subpopulations were inferred and annotated by identifying differentially expressed marker genes with log-fold change higher than 0.4 using MAST in FindAllMarkers function. The pro/anti-inflammation ratio in the macrophage population was calculated as the fold changes of proportions between the inferred pro-vs. anti-inflammatory clusters. For the identified CD8⁺ T-cell subpopulations, we calculated the score of Tcf1+ stem-like signature for each cell by using Seurat's AddModuleScore function based on the gene sets previously reported (Siddiqui et al., 2019).

Analysis of scTCR-Seq Data

Alignment to the GRCm38 reference genome and TCR contig annotation were performed by Cell Ranger vdj pipeline (10× Genomics, v2.1.0). For the TCR clonotype analysis, only cells assigned with both productive TRA and TRB sequences were kept for further analysis. If one cell had two or more TRA-TRB pairs identified, the pair with higher UMIs was considered as the dominant TRA-TRB pair in the corresponding cell and used in the analysis. We defined each unique TRA-TRB pair as a clonotype. The clonal status of TCR clones were characterized as non-clonal (n=1) and clonal (n≥2) based on their cell numbers. The TCR clonotype of each cell was further linked to inferred functional subsets based on the barcode information. We used STARTRAC package to calculate the expansion and transition indices of distinct T-cell subsets.

### Bulk Tumor TCR-Seq Data Analysis

As reported previously (10), raw reads were submitted to the QIAGEN GeneGlobe Data Analysis Center (qiagen.com/us/shop/genes-and-pathways/data-analysis-center-overview-page/) to estimate the abundance of reads of unique CDR3 sequence and generate TCR clonotype calls. R package tcR (Nazarov et al., 2015) was used to perform all the statistical analysis for TCR repertoires, including: i) size of large clones with frequency not less than 5%; ii) diversity estimates using ecological diversity and Gini-Simpson indices, and iii) common TCR repertories (identified based on unique alpha or beta chains' CDR3 sequences) shared by distinct tumor-involved regions within an organ tissue or by different organ tissues.

### Statistical Analysis of Non-Clinical Data

No statistical methods were used to predetermine sample size. We used the paired t-test to determine the statistical significance of differences between two variables. Survival curves were compared via the logrank test. We applied the linear mixed effects model on the log₁₀-transformed BLI intensity data. In this model, treatment group, days, and treatment*day interactions were treated as fixed effects, while a random intercept and time slope were assumed for individual mouse. Pairwise comparisons between treatment regimen groups were conducted within the mixed model framework with Bonferroni correction for multiple testing. All other statistical analyses were carried out using R and GraphPad Prism 7.

### Results

### Prior ICT Enhances MAPKi Responses in Melanoma Patients

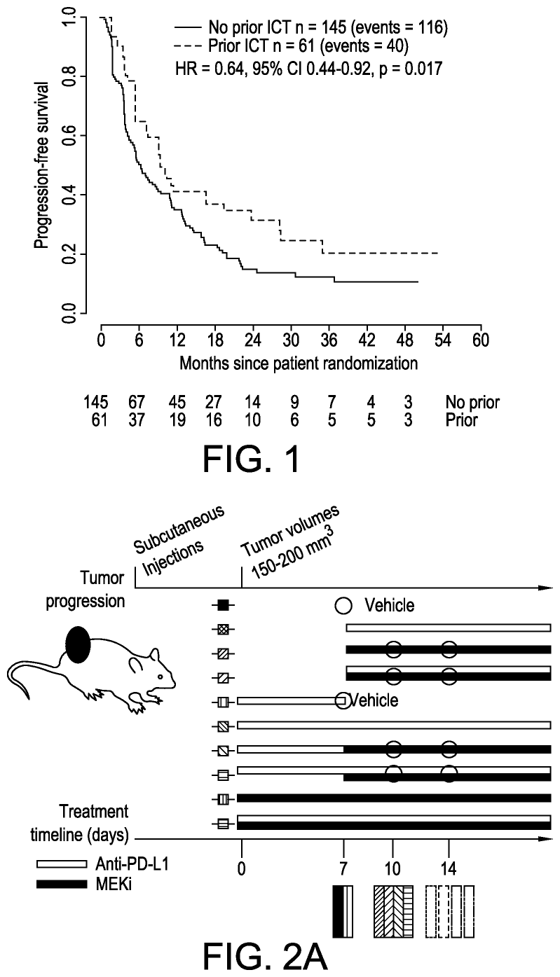

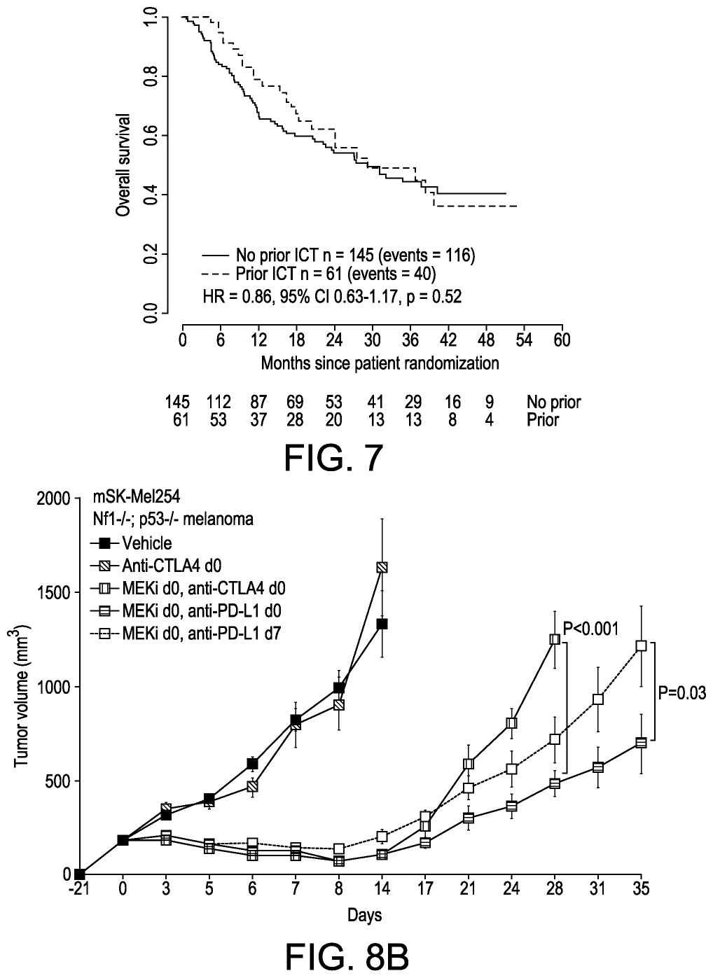

In a trial of the MEKi binimetinib vs. dacarbazine for patients with NRAS^{MUT} melanoma, greater clinical benefit (median progression-free survival or PFS, confirmed overall response, and median duration of objective response) of binimetinib was associated with prior ICT (Dummer et al., 2017). S1320 was a phase 2 randomized clinical trial comparing intermittent (n=101) versus continuous (n=105) dosing of the BRAFi dabrafenib and MEKi trametinib for BRAF^{V600E/K} metastatic melanoma. The trial randomized patients between 2013 and 2019. After an eight-week lead-in period of continuous treatment, patients who did not progress were randomized to either continuous or intermittent dosing of both drugs on a 3-week-off, 5-week-on schedule. As reported (Algazi et al., 2020), PFS was longer with continuous vs. intermittent dosing. Prior exposure to ICT was a randomization stratification factor in the trial, with 61 patients (30%) with prior exposure (anti-CTLA-4 alone, n=21; anti-PD-1 alone, n=22; anti-CTLA-4 and anti-PD-1 separately, n=6; anti-PD-1 alone and anti-PD-1+anti-CTLA-4 separately, n=1; anti-PD-1, anti-CTLA-4, anti-PD-1+anti-CTLA-4 separately, n=1; unknown, n=4). Patient characteristics were similar between patients who did and did not have prior ICT (Table 1). PFS was longer among patients with prior ICT on univariate (hazard ratio (HR)=0.64, 95% confidence interval (CI) 0.44-0.92, p=0.017) and multivariable analysis (HR=0.60, 95% CI 0.47-0.77, p=0.009; FIG. 1; Table 2A). There were no significant differences in overall survival (OS) by prior ICT on univariate (HR=0.86, 95% CI 0.54-1.35, p=0.51) or multivariable analysis (HR=0.85, 95% CI 0.63-1.17, p=0.52; FIG. 7, Table 2B). There was no evidence of heterogeneity in these results by treatment arm (interaction p-value=0.55 for PFS, p=0.67 for OS).

### Anti-PD-1/L1 Lead-In Preceding MAPKi Combination Optimizes Antitumor Efficacy

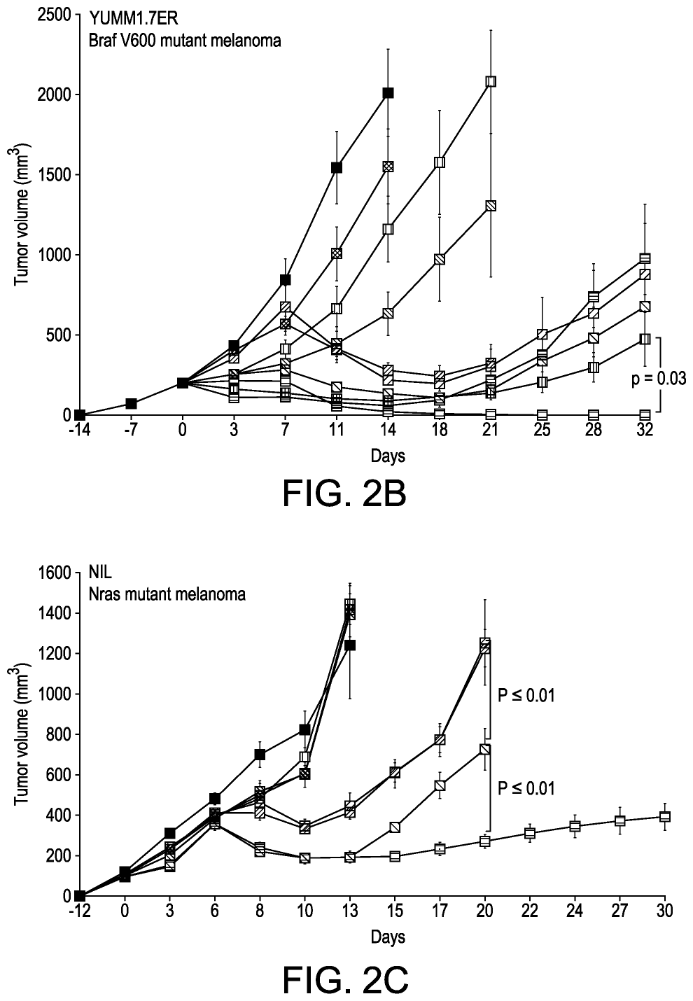

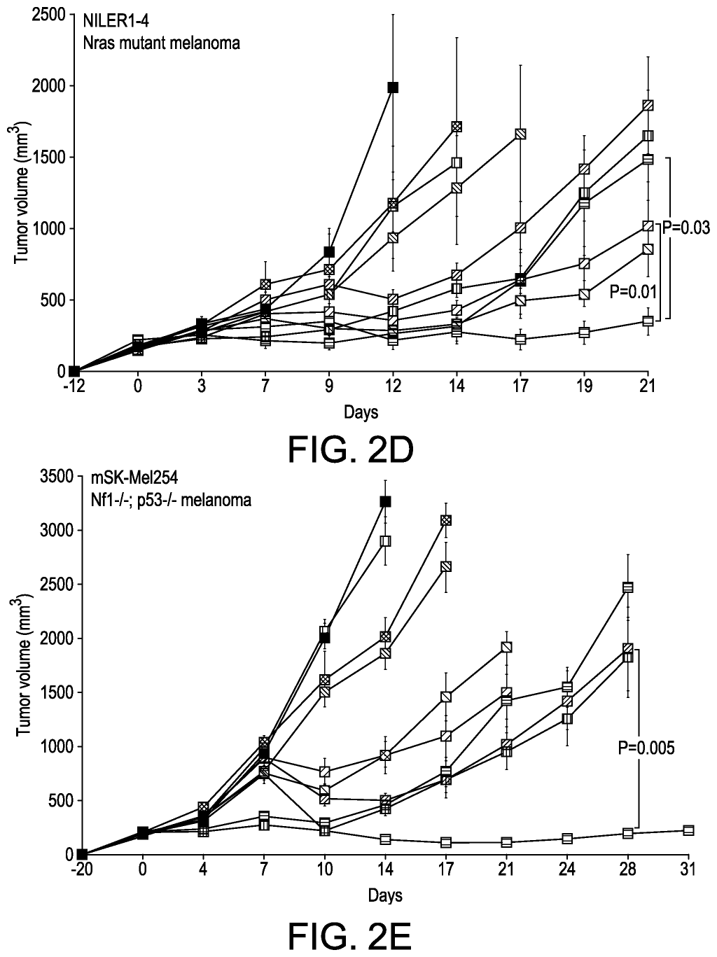

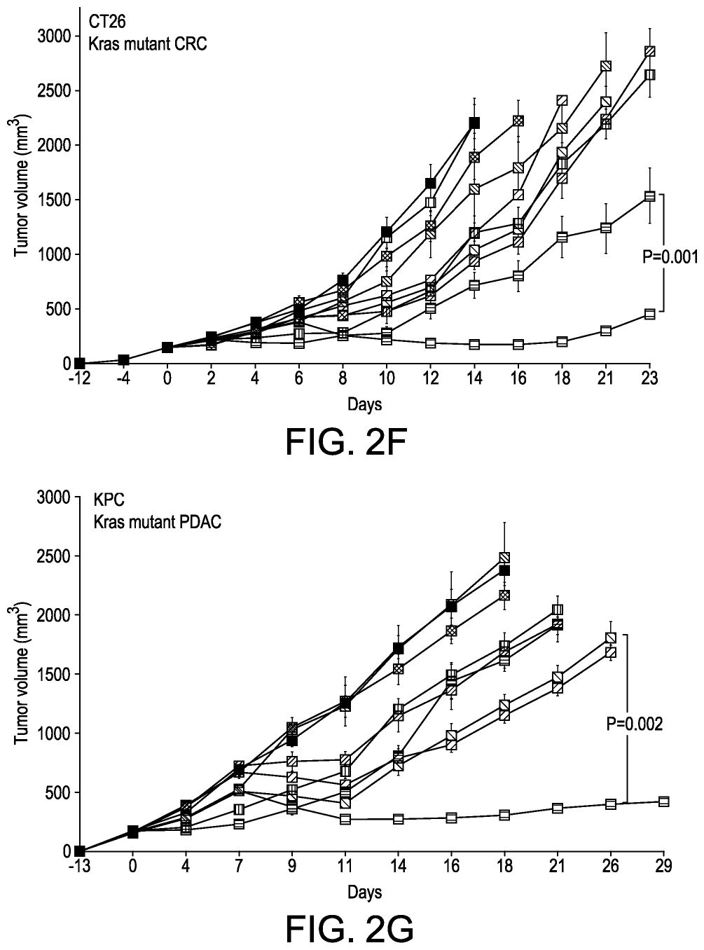

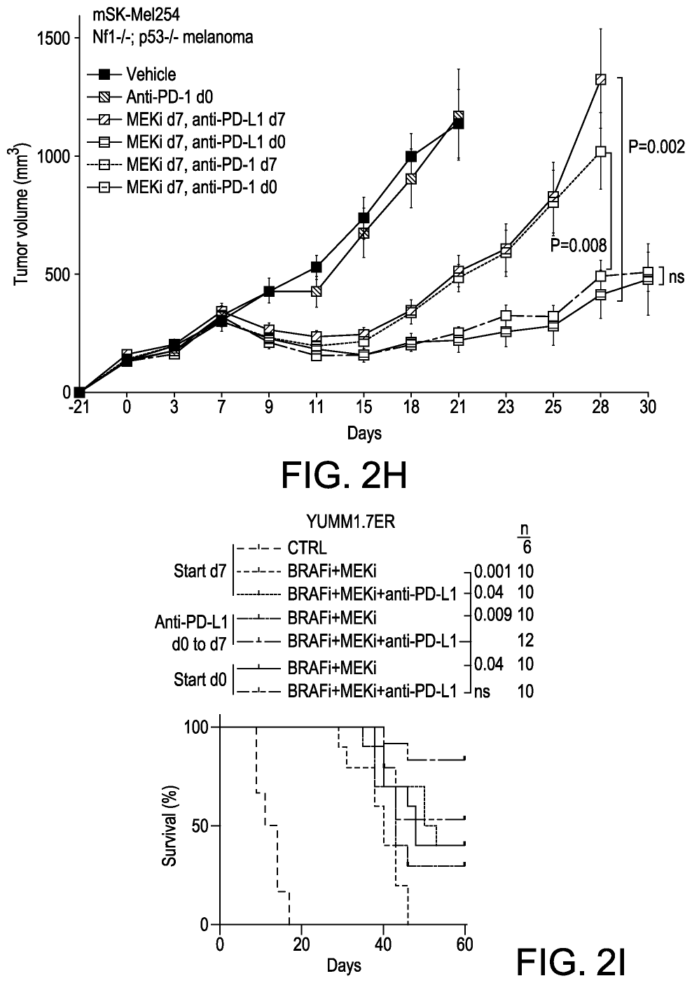

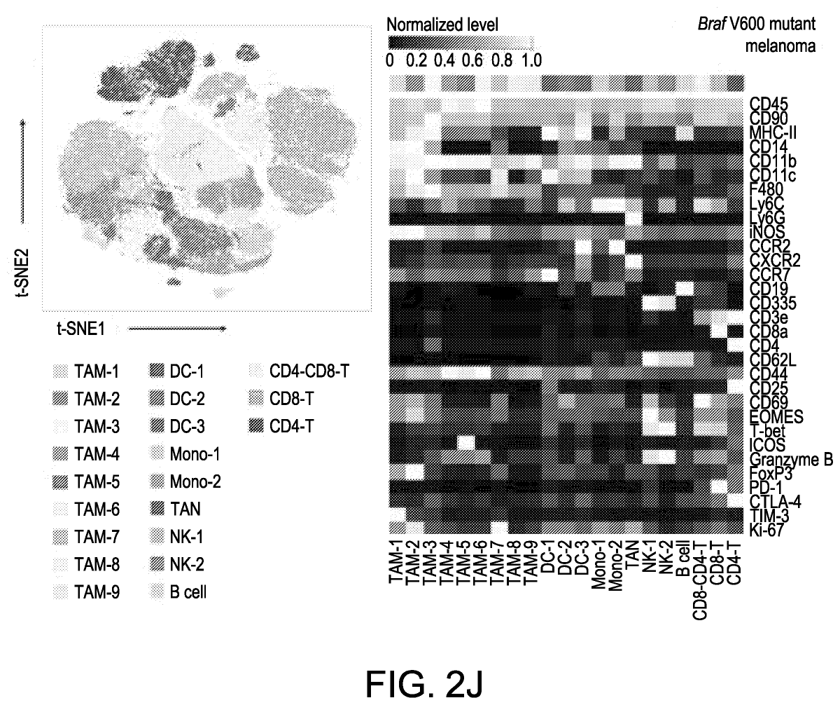

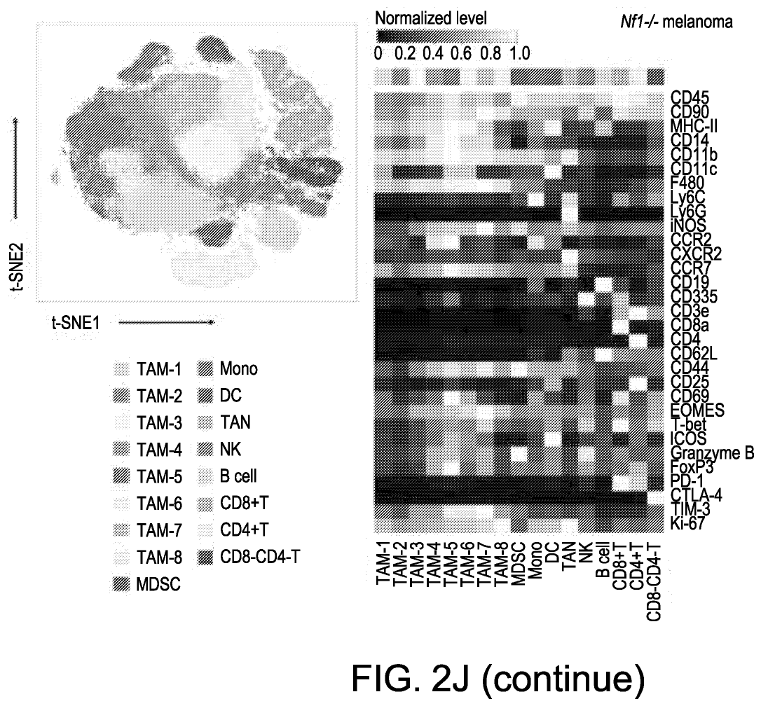

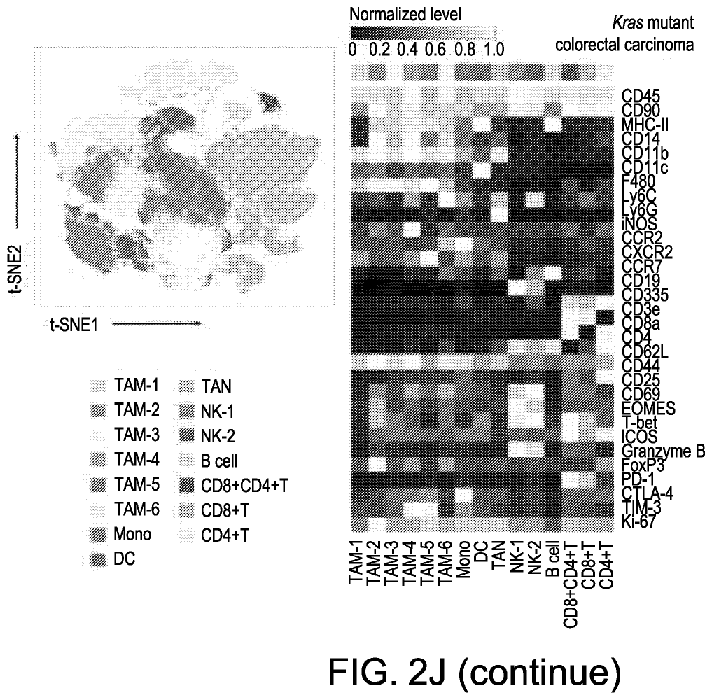

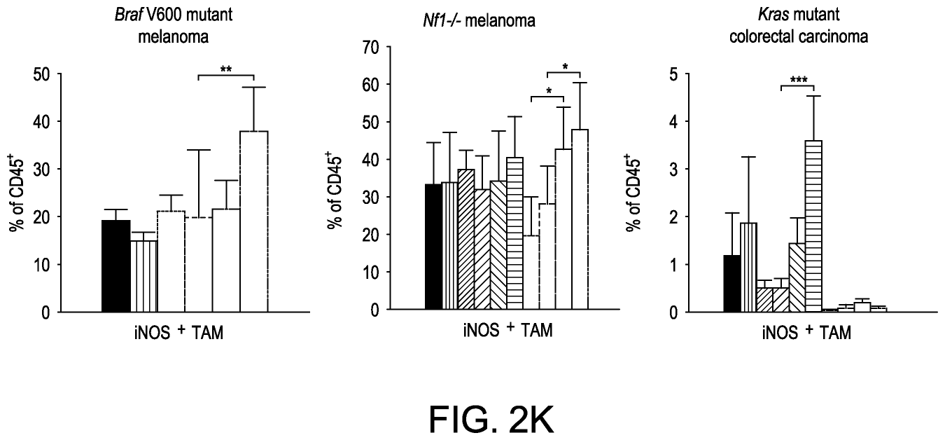

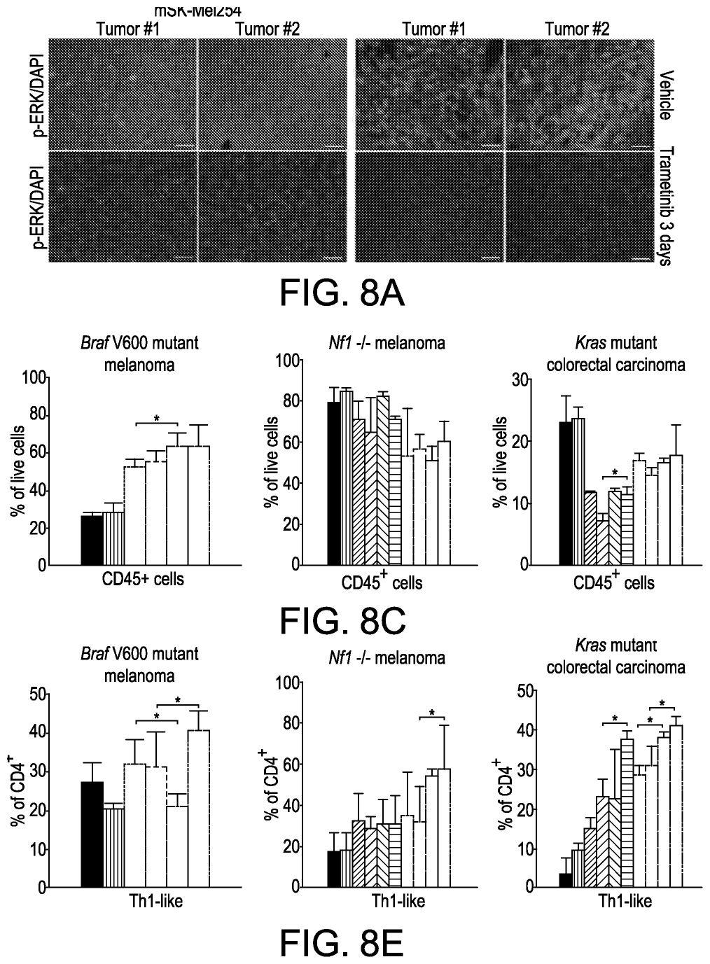

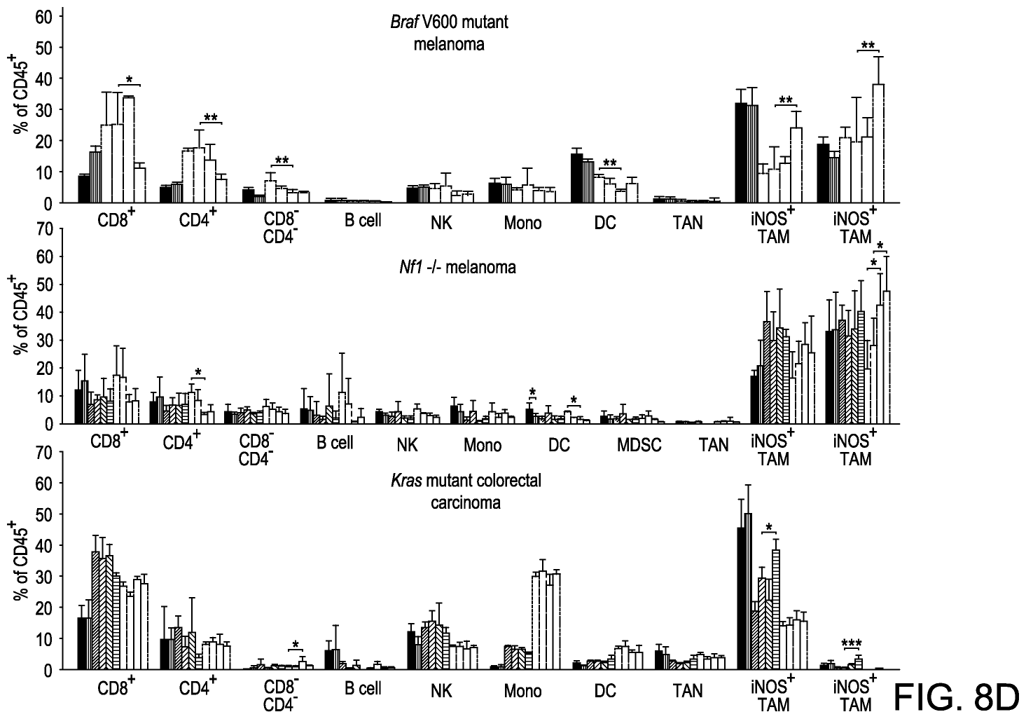

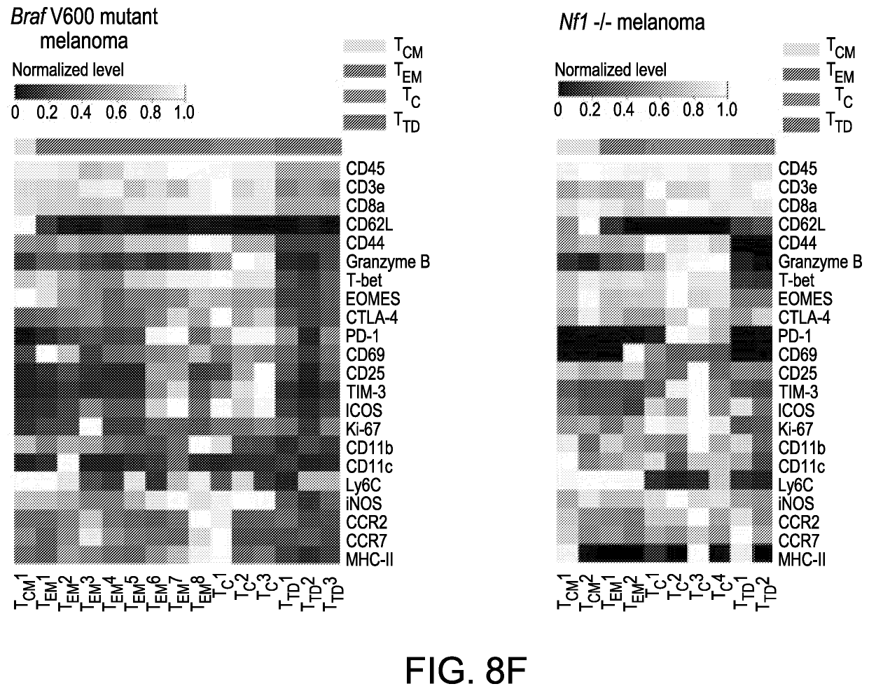

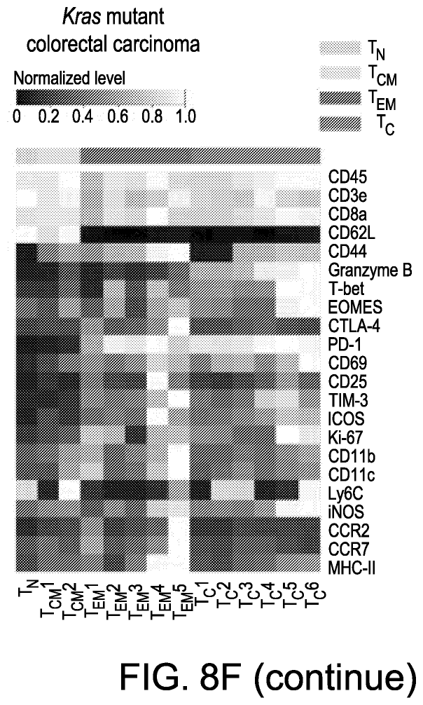

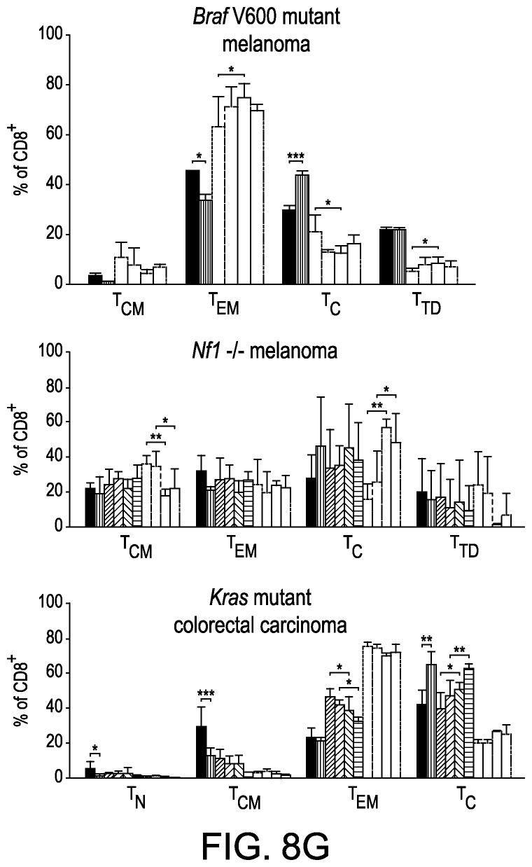

Using syngeneic subcutaneous tumor models, we tested whether brief anti-PD-L1 (or anti-PD-1) pretreatments (two doses over one week) can improve subsequent responses to MEKi, with or without continuing anti-PD-L1 dosing with MEKi. We defined time at which the average tumor volumes reach 120-140 mm³ as day 0 (d0) and do to d7 as the anti-PD-1/L1 lead-in period (FIG. 2A). We treated tumor-bearing mice in the following control (no active treatment, single-agent treatment, or simultaneous combination treatment) groups: (i) vehicle (starting on d7), (ii) anti-PD-L1 (starting on d0, d7 or from do to d7 only), (iii) MEKi (starting on do or d7), and (iv) MEKi+anti-PD-L1 (simultaneously starting on d0 or d7). We expected superior antitumor activity with anti-PD-L1 lead-in before MEKi dosing in two experimental groups: (i) MEKi d7, anti-PD-L1 d0 to d7 (anti-PD-L1 started on do and stopped on d7 followed by MEKi started on d7), and (ii) MEKi d7, anti-PD-L1 d0 (anti-PD-L1 started on do followed by MEKi started on d7, while continuing anti-PD-L1) (FIG. 2A). Trametinib dose level for each tumor model was chosen based on the minimal dose required to elicit tumor stabilization or regression early on-treatment and near complete p-ERK suppression on day 3 (FIG. 8A). We used six murine syngeneic tumor models: (i) Braf^{V600E} melanoma with high mutational burden (YUMM1.7ER; FIG. 2B) (Wang et al., 2017), (ii) Nras^{Q61R} melanoma (NIL; FIG. 2C) (Hong et al., 2018), (iii) Nras^{Q61R} melanoma with high mutational burden (NILER1-4; FIG. 2D) (Hong et al., 2021), (iv) Nf1^{-/-} melanoma (mSK-Mel254; FIG. 2E), (v) Kras^{G12C} colorectal carcinoma (CT26; FIG. 2F), and (vi) Kras^{G12C} pancreatic adenocarcinoma (KPC; FIG. 2G).

In all models, anti-PD-L1 treatment alone (d0, d0 to d7, or d7) had minimal (YUMM1.7ER, NILER1-4, mSK-Mel254) to no (NIL, CT26, KPC) tumor growth-inhibitory effect (FIGS. 2B to 2G). In all models (FIGS. 2B to 2G), at the trametinib doses chosen, treatment with MEKi d7 vs. MEKi d7, anti-PD-L1 d7 elicited only transient tumor regression and small differences, if any, in the average tumor volumes over time. In general (FIGS. 2B and FIGS. 2D to 2G), MEKi d0 or MEKi d0, anti-PD-L1 d0 (i.e., treatments started on smaller tumors) still did not improve the durability of antitumor activities, except in CT26 (MEKi d0, anti-PD-L1 d0 vs. MEKi d7, anti-PD-L1 d7; p<0.008). In one tumor model (mSK-Mel254) (FIG. 8B), using MEKi started on do against smaller tumors, we observed that the efficacies of regimens MEKi d0, anti-CTLA-4 d0 and MEKi d0, anti-PD-L1 d7 were lower than that of the regimen MEKi d0, anti-PD-L1 d0. Thus, anti-CTLA-4 and delayed dosing of anti-PD-L1 (vs. MEKi) were not studied further in subcutaneous tumor models. Importantly, between the two regimens we hypothesized to elicit the most robust antitumor activity, anti-PD-L1 lead-in followed by MEKi combination consistently led to the most extensive and durable tumor regression (FIGS. 2B to 2G). This regimen was either not associated with body weight loss or associated with a weight loss that was <10% compared to any other concurrent regimen. In contrast, the efficacy of the regimen of MEKi d7, anti-PD-L1 do to d7 was not superior to the efficacies of the regimens MEKi d7 or MEKi d7, anti-PD-L1 d7. Thus, brief dosing with anti-PD-L1 prior to its combination with MEKi in MAPK-addicted tumor models overcame anti-PD-L1 innate resistance and delayed acquired MEKi resistance.

Using mSK-Mel254, we tested whether anti-PD-1 would produce a similar priming effect (FIG. 2H). As with anti-PD-L1 d0 (FIG. 2E), anti-PD-1 d0 yielded little to no tumor growth inhibition. Clearly, the regimen of MEKi d7, anti-PD-1 d0 was superior to the regimen of MEKi d7, anti-PD-1 d7 (FIG. 2H). Using YUMM1.7ER, we also tested regimens containing the combination of BRAFi+MEKi. We started BRAFi+MEKi+anti-PD-L1 treatments either on d0 or d7. For the latter group (starting on d7), we tested the impact of anti-PD-L1 lead-in (2 doses). Consistent with previous data using MEKi alone (FIG. 2B), anti-PD-L1 lead-in followed by BRAFi+MEKi combination consistently led to the most durable antitumor activity (FIG. 2I), which was associated with mice weight loss that was <10% compared to any other concurrent regimen.

Pro-Inflammatory TAM Polarization Distinguishes the Tumor Microenvironment after Sequential-Combinatorial Therapy

To identify immune cell alterations specifically elicited by the regimen of anti-PD-L1 lead-in prior to MEKi combination, we sampled tumors (n=3-4/group) with vehicle or anti-PD-L1 (priming) treatments on d7 and tumors (n=3-4/group) on d10 and d14 in four regimens: (i) MEKi d7. (ii) MEKi d7, anti-PD-L1 d7, (iii) MEKi d7, anti-PD-L1 d0 to d7, and (iv) MEKi d7, anti-PD-L1 d0 (FIG. 2A). Dissociated cells from three tumor models (YUMM1.7ER, mSK-Mel254, CT26) were subjected to analysis by an immune cytometry by time-of-flight (CyTOF) panel published previously (Hong et al., 2021). We found that the three tumor models were highly distinct in the levels of infiltration by CD45⁺ cells, which ranged from 20-80% without treatment or after two doses of anti-PD-L1 treatments (FIG. 8C). These percentages increased (YUMM1.7ER), decreased (mSK-Mel254), or decreased and then increased (CT26) over time after MEKi-containing treatments (FIG. 8C). Among CD45⁺ cells, two doses of anti-PD-L1 increased the abundance of CD8⁺ T cell (vs. vehicle) only in YUMM 1.7ER tumors (FIG. 8D). After MEKi-containing treatments (vs. vehicle treatment), the percentages of CD8⁺ T cells among CD45⁺ cells increased only in YUMM1.7ER and CT26, but these increases did not associate with treatment efficacy (FIG. 8D).

Among CD45⁺ cells in all treatment groups, tumor-associated macrophages (TAMs) were the most abundant subpopulation across the three tumor models (FIGS. 2J and 8D). We identified 6-9 TAM subpopulations across the three tumor models (FIG. 2J). iNOS-high or iNOS⁺ M1-like TAMs were identified in 6 of 9 and 6 of 8 TAM subpopulations in YUMM 1.7ER and mSK-Mel254 but only in 1 of 6 TAM subpopulations in CT26 (FIG. 2J). Despite these differences, we observed specific induction of iNOS⁺ M1-like TAMs only in the regimen of MEKi d7, anti-PD-L1 d0 (FIG. 2K). Moreover, after sub-clustering CD4⁺ T-cells, we found that Th1-like (T-bet high) CD4⁺ T cells were also specifically induced only in the regimen of MEKi d7, anti-PD-L1 d0 (FIG. 8E). After sub-clustering CD8⁺ T-cells, we found that two doses of anti-PD-L1 elevated the levels of granzyme B-high CD8⁺ cytotoxic T-cells (T_{C}) (FIGS. 8F and 8G). However, this elevation was not maintained after initiating any MEKi-containing regimen (YUMM1.7ER) or not specifically maintained in the most efficacious regimen (mSK-Mel254, CT26) (FIG. 8G).

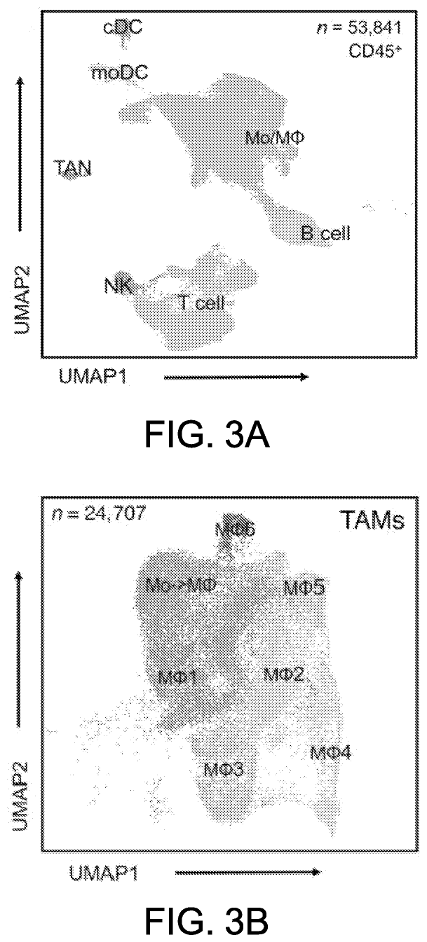

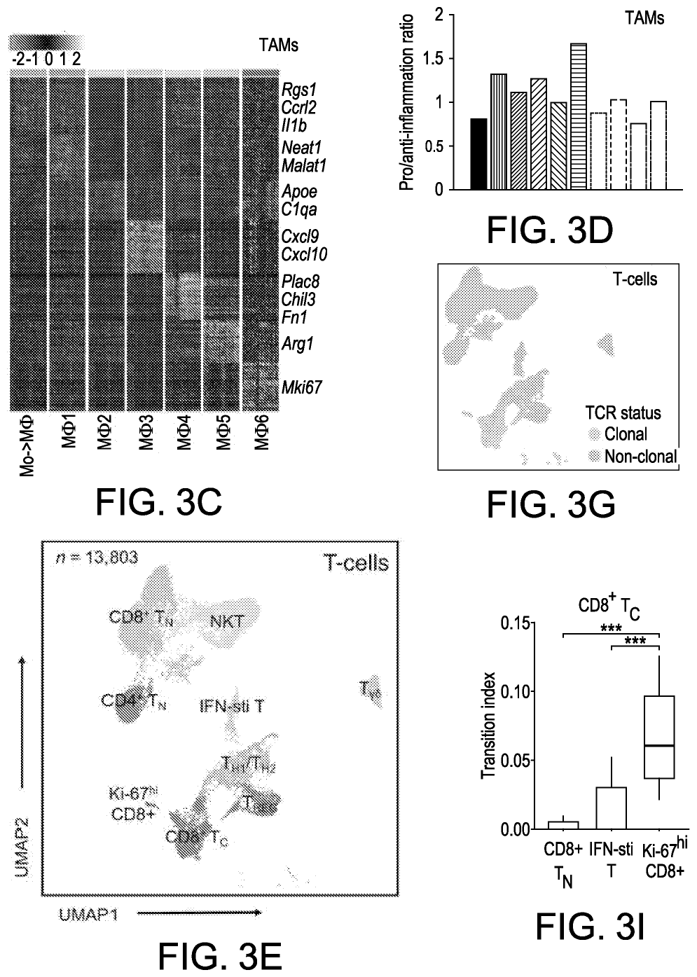

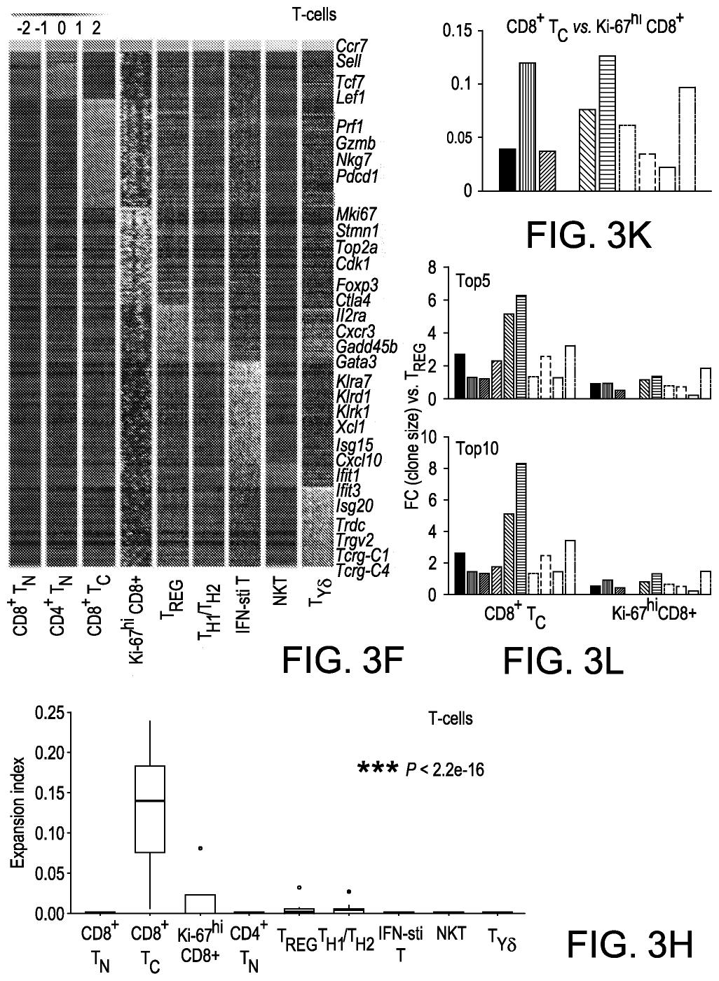

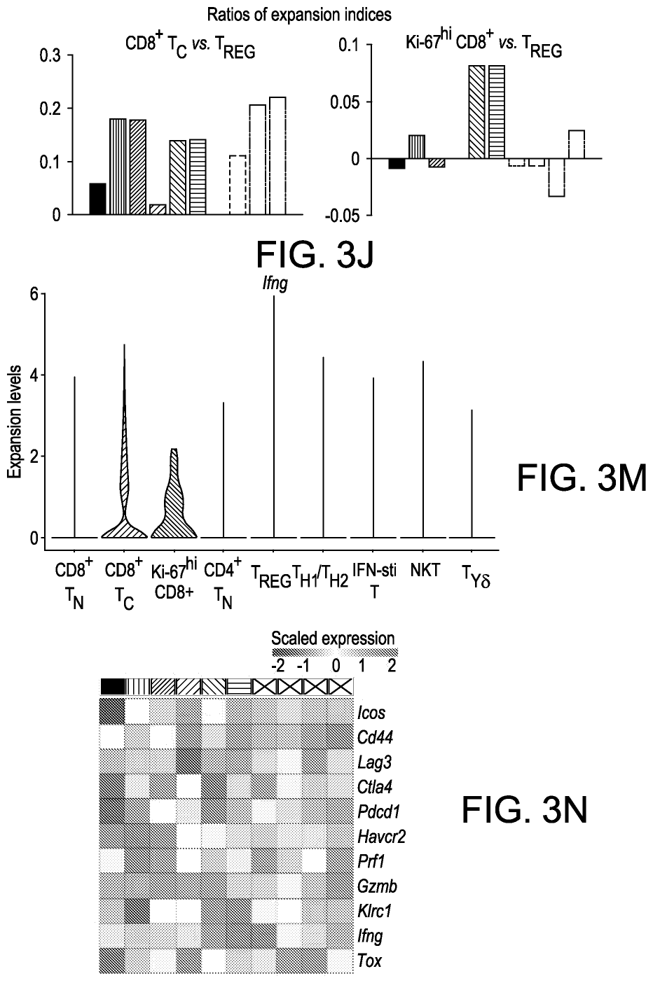

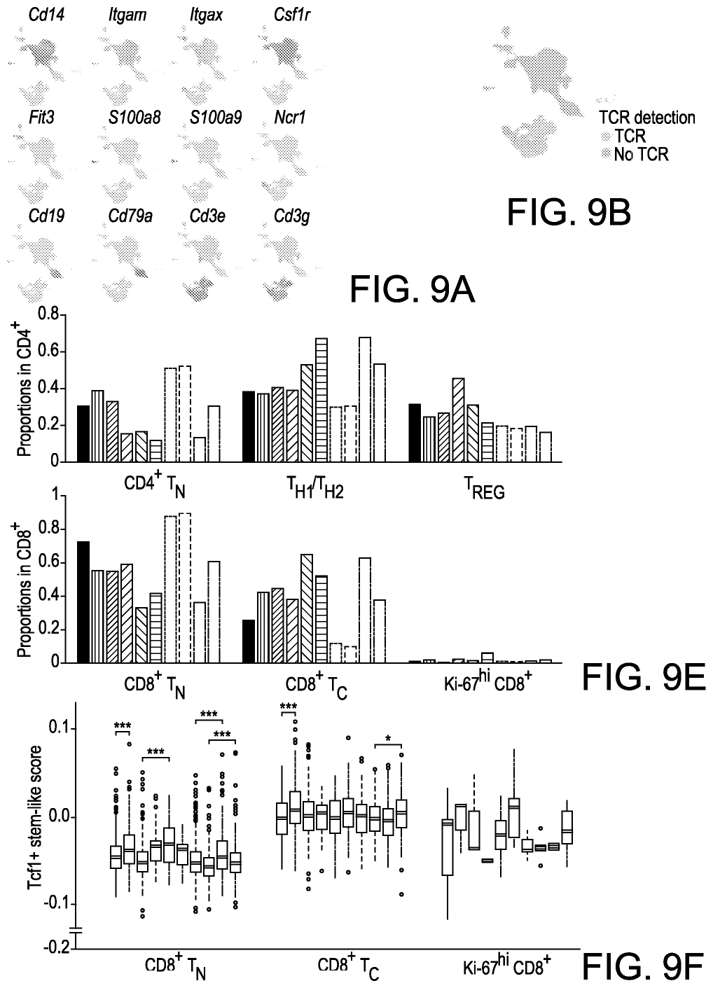

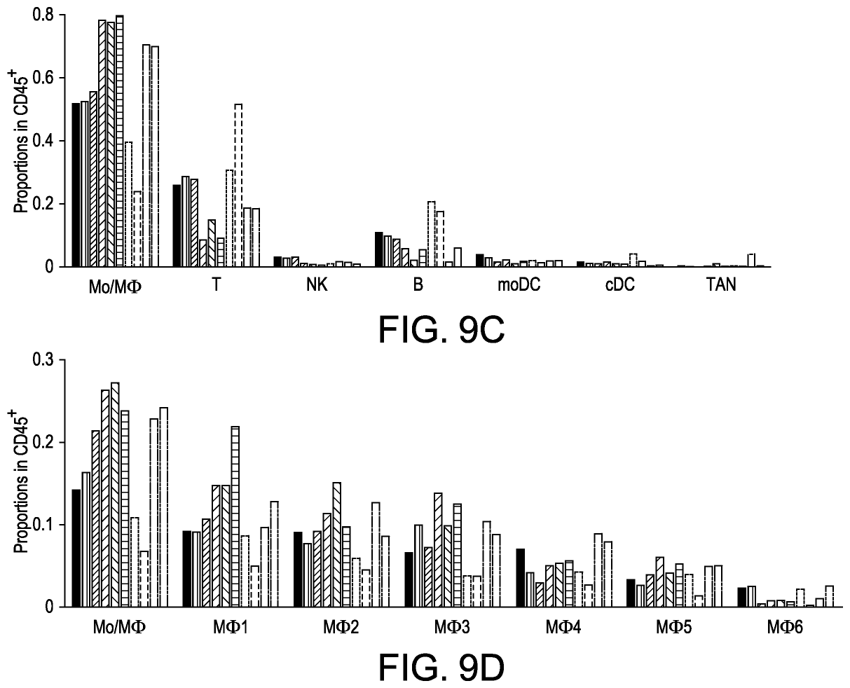

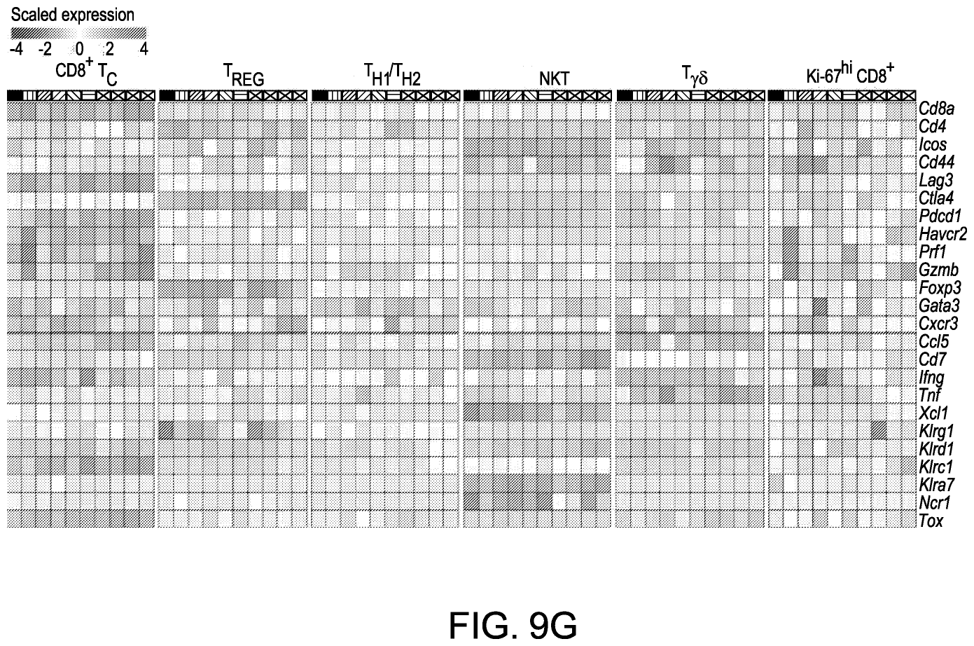

We sought to corroborate TAM findings above (FIG. 2K) at the transcriptomic level. Sorted CD45⁺ cells from four mSK-Mel254 tumors per regimen, per time point (FIG. 2A) were admixed and then subjected to coupled 5′ single-cell RNA-sequencing (scRNA-seq) and T-cell receptor-sequencing (scTCR-seq). In total, data from 53,841 CD45⁺ cells passed quality control. Based on expression profiles of known lineage marker genes, we annotated seven major immune cell types, including monocyte/macrophages (Mo/MΦ), T cells, B cells, natural killer (NK) cells, tumor-associated neutrophils (TANs), and two dendritic cell (DC) subsets (monocyte-derived DC and classical DC) (FIGS. 3A, 9A and 9B). Consistent with CyTOF analysis (FIGS. 2J and 8D), TAMs constituted the most abundant CD45⁺ subpopulation, followed by T cells (FIG. 9C). Re-clustering the monocyte/macrophage population identified seven sub-populations (FIGS. 3B, 3C and 9D). Cells in MΦ1 and MΦ3 displayed significant upregulation of pro-inflammatory genes (e.g., Neat1, Malat1, Cxcl9, and Cxcl10). MΦ2, MΦ4, and MΦ5 subpopulations upregulated anti-inflammatory genes (e.g., Apoe, C1qa, Chil3, and Arg1). One subpopulation (Mo→MΦ) highly-expressed Ccrl2, Il1b, and Rgs1, suggesting monocytes transitioning into to macrophages. We then calculated the pro-vs. anti-inflammatory ratios and found that two lead-in doses of anti-PD-L1 (vs. vehicle treatment) enhanced this ratio from <1 to >1 (FIG. 3D). However, this enhanced ratio was reversed subsequently with any MEKi-containing regimen, except the most efficacious regimen (MEKi d7, anti-PD-L1 d0), which further enhanced this ratio on day 10 (FIG. 3D).

Activated, Proliferative, and Cytolytic CD8+ T-Cell Gene Expression Tracks with Optimized Regimen

We next analyzed the T-cell population (n=13,803 cells) using coupled scRNA- and scTCR-seq. By sub-clustering, we identified nine T-cells subpopulations (FIGS. 3E and 3F). These included three CD8⁺ subpopulations (naïve, T_{C} and Ki-67^{hi}); three CD4⁺ subpopulations (naïve, regulatory or T_{REG}, T helper 1/2 or Th1/h2 that co-expressed Th1 and Th2 genes such as Cxcr3 and Gata3), NK T cells or NKTs, interferon (Ifn)-stimulated T cells, and gamma-delta T cells (T_{γδ}). As predicted, two lead-in doses of anti-PD-L1 elevated CD8⁺ T_{C} levels (FIG. 9E). Importantly, these elevated CD8⁺ T_{C} levels were either maintained or surpassed with subsequently switching to or combining with MEKi (FIG. 9E). By d14 in tumors treated with MEKi d7 or MEKi d7, anti-PD-L1 d7, CD8⁺ T_{C} levels dropped clearly below that in the vehicle-treated tumors.

Among 7,017 TCR clonotypes identified with unique a and b chain pairs, 350 were represented by two or more cells, which resulted in 1,384 clonal T cells (FIG. 3G). We calculated the expansion indices across all nine T-cell subpopulations and observed that CD8⁺ T_{C} cells harbor the highest degree of clonal expansion, followed by the Ki-67^{hi} CD8⁺ T cells (FIG. 3H and Table 3). Consistently, clonal T cells were concentrated in the T_{C} subpopulation (FIGS. 3E and 3G). Interestingly, transition index analysis associated Ki-67^{hi} CD8⁺ T cells with T_{C} subpopulations (FIG. 3I), suggesting a differentiation trajectory from proliferative CD8⁺ T cells to the clonally expanded T_{C} subpopulation. We then calculated the ratios of expansion indices of CD8⁺ T_{C} or Ki-67^{hi} CD8⁺ T cells to T_{REG} to estimate the net antitumor status in each regimen and time point. Two lead-in doses of anti-PD-L1 increased both ratios, the elevation of which was either maintained or surpassed after MEKi combination (FIG. 3J). In tumors treated with MEKi d7 or MEKi d7, anti-PD-L1 d7 (i.e., no prior priming doses of anti-PD-L1), these ratios persisted at low levels near that observed in the vehicle-treated group. When we examined the transition indices of CD8⁺ T_{C} from Ki-67^{hi} CD8⁺ T cells over time, we observed increased transition after anti-PD-L1 lead-in doses, and this transition was maintained and highest at both subsequent time points in and only in tumors on the regimen of MEKi d7, anti-PD-L1 d0 (FIG. 3K).

To corroborate the specific association of CD8⁺ T_{C} and Ki-67^{hi} T cells with the most efficacious regimen, we analyzed the largest (i.e., most expanded) TCR clonotypes (Table 3), stemness, or expression of specific functional genes. The fold changes in the total clone size of top 5 or top 10 TCR clonotypes of either CD8⁺ T_{C} or Ki-67^{hi} T cells were calculated compared to that of CD4⁺ T_{REG} cells. This revealed that the most efficacious regimen yielded the highest expansion of top T_{C} clonotypes (vs. T_{REG}) at both time points and of top Ki-67^{hi} CD8⁺ T cell clonotypes (vs. T_{REG}) at the last time point (FIG. 3L). Tcf1⁺ stem-like CD8⁺ T cells are thought to be critical for tumor control in response to ICT (Siddiqui et al., 2019). Hence, we scored CD8⁺ T cell subpopulations for enrichment of this signature across regimens and time points (FIG. 9F). For the Ki67^{hi} CD8⁺ T cell subpopulation, this score trended higher (vs. vehicle treatment) after two anti-PD-L1 lead-in doses and stayed high on d10 only in the most efficacious regimen. By d14, this score dropped across all MEKi-containing regimens but remained highest in the tumors from the most efficacious regimen. Moreover, we visualized the expression levels of functional genes across T-cell subpopulations (FIG. 9G). We noted that the critical gene, Ifng, was expressed (FIGS. 3M and 9G) by most CD8⁺ T_{C} and Ki-67^{hi} T cells, supportive of their antitumor functional importance. Critically, within the T_{C} subpopulation, two anti-PD-L1 lead-in doses upregulated the expression of activation genes (Cd44), inhibitory genes (Lag3, Ctla4, Havcr2), cytolytic genes (Prf1, Gzmb), and effector genes (Ifng) (FIG. 3N). On d10, treatment on the most efficacious regimen led to the highest levels of activation/exhaustion gene (Pdcd1) and Ifng. By d14, treatment on the most efficacious regimen led to the highest expression of both Pfr1 and Gzmb and the highest ratio of Pdcd1 to Tox, the master regulator of T-cell exhaustion. Interestingly, MEKi monotherapy induced the highest levels of Ctla-4 at both time points (FIG. 3N), suggesting anti-CTLA-4 antibody as a combinatory agent. Foxp3 expression was down-regulated in T_{REG} specifically in the most efficacious regimen (FIG. 9G).

### Functional Contributions of TAM Polarization and CD8+ T Cells

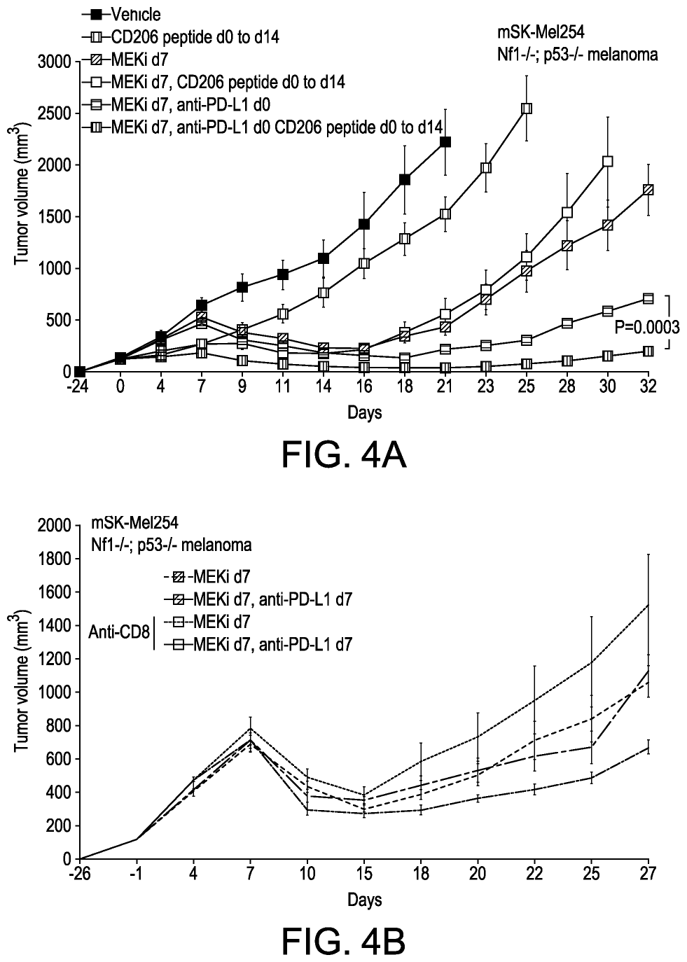

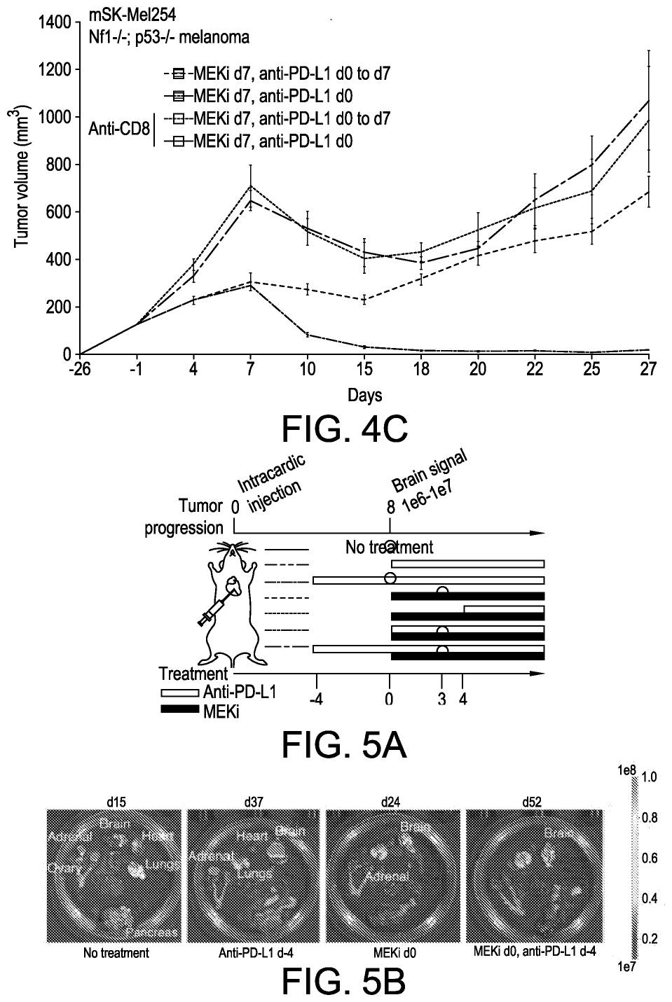

We assessed whether targeting of M2-like TAMs by a peptide agonist of CD206 (Ghebremedhin et al., 2020; Jaynes et al., 2020) would augment the priming effects of anti-PD-L1 (FIG. 4A). Using the mSK-Mel254 model, we observed that the CD206 agonist, RP832c, on its own (dosed from do to d14) elicited a small degree of growth inhibition, but, on top of MEKi d7, CD206 peptide do to d14, elicited no priming effect (FIG. 4A). However, combining the CD206 agonist with anti-PD-L1 improved priming, as the regimen of MEKi d7, anti-PD-L1 d0, CD206 peptide do to d14 elicited deeper and more durable tumor regression when compared to the regimen of MEKi d7, anti-PD-L1 d0 (FIG. 4A). We also tested using mSK-Mel254 whether neutralizing CD8⁺ T cells systemically would diminish or abolish the priming effect of two doses of anti-PD-L1. In the context of two regimens (MEKi d7; MEKi d7, anti-PD-L1 d7) without anti-PD-L1 lead-in (FIG. 4B), CD8⁺ T-cell neutralization had no impact on tumor regression or resistance development. This lack of effect is consistent with the lack of additivity when MEKi is initiated with anti-PD-L1 on established (700-900 mm³) tumors (FIGS. 2B to 2G). Moreover, when we neutralized CD8⁺ T cells, we reversed the growth deceleration effect elicited by the two priming anti-PD-L1 doses from do to d7 (FIG. 4C). Importantly, while CD8⁺ T-cell neutralization did not change the onset of acquired resistance in the regimen MEKi d7, anti-PD-L1 d0 to d7, it abolished the benefit (i.e., durability of tumor regression) of the regimen MEKi d7, anti-PD-L1 d0 (FIG. 4C).

### PD-1/L1 Plus CTLA-4 Blockade Before MAPKi Combination Prolongs MBM Suppression and Survival of Mice

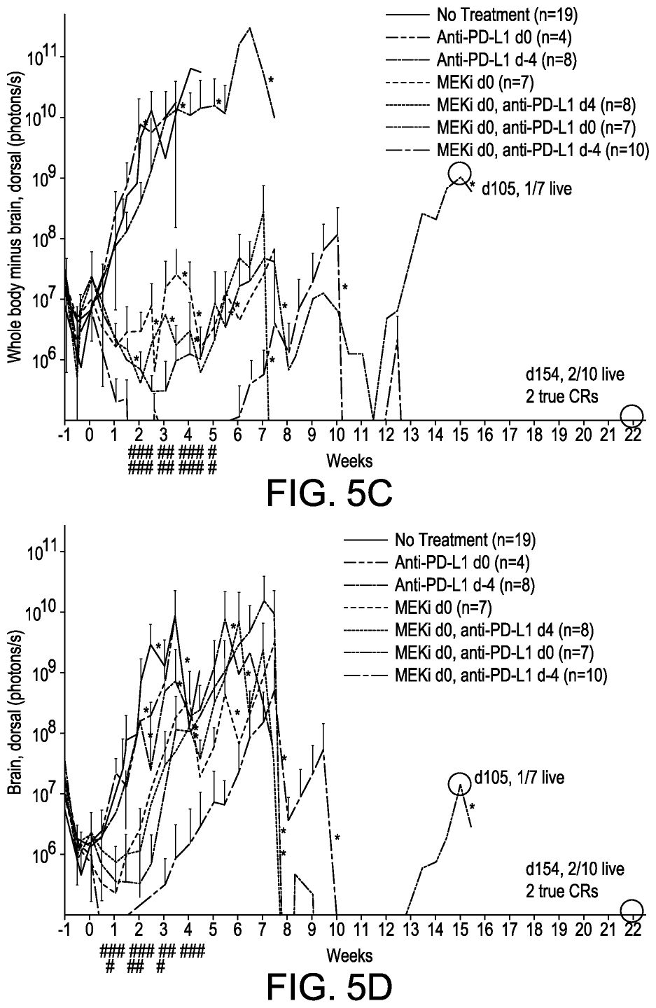

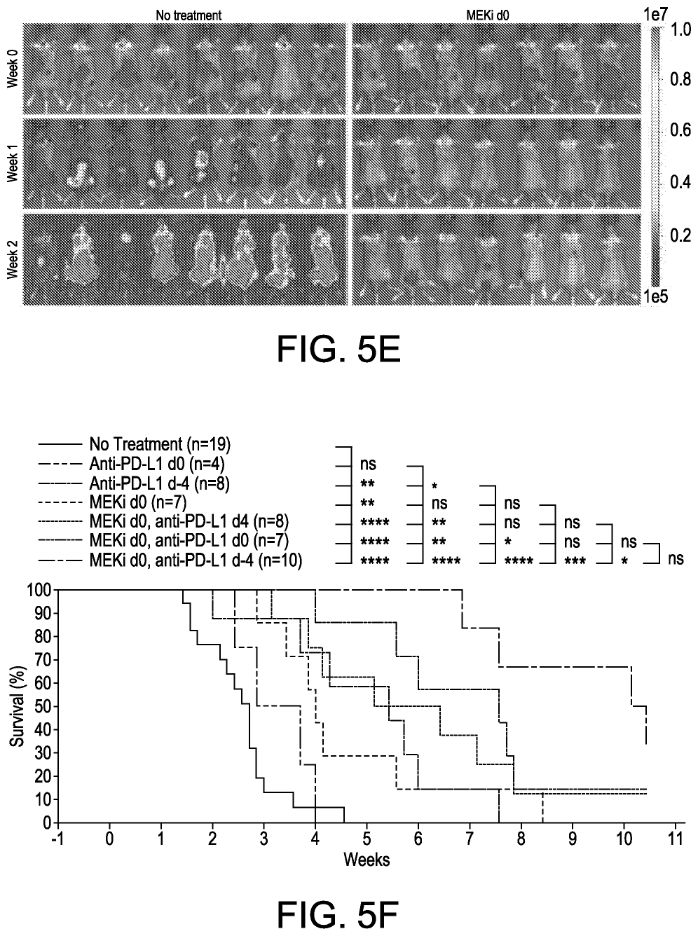

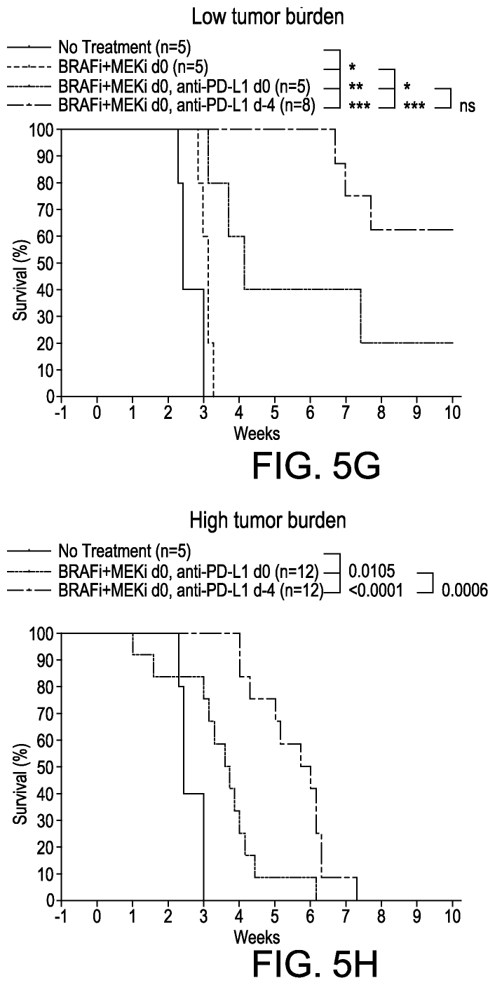

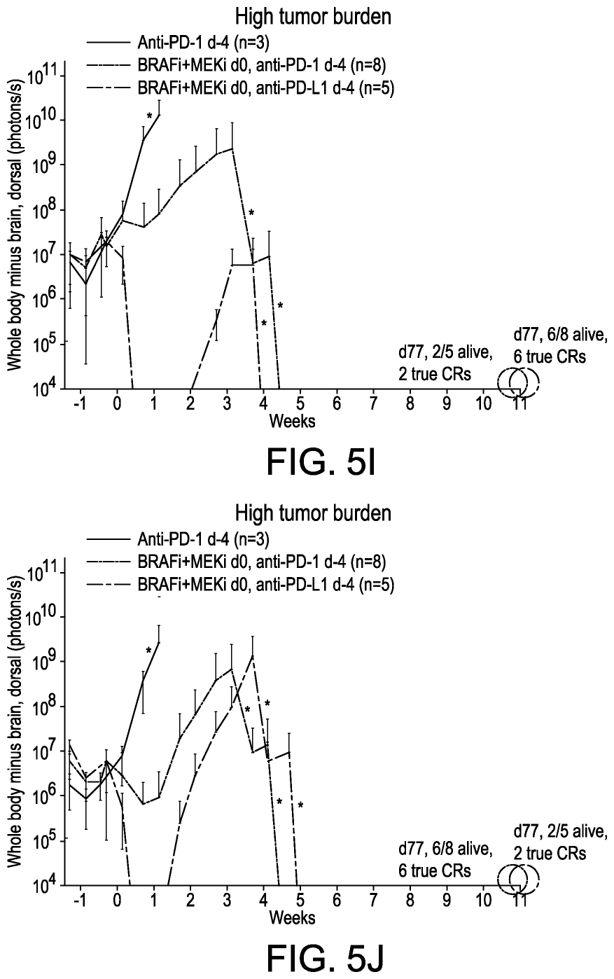

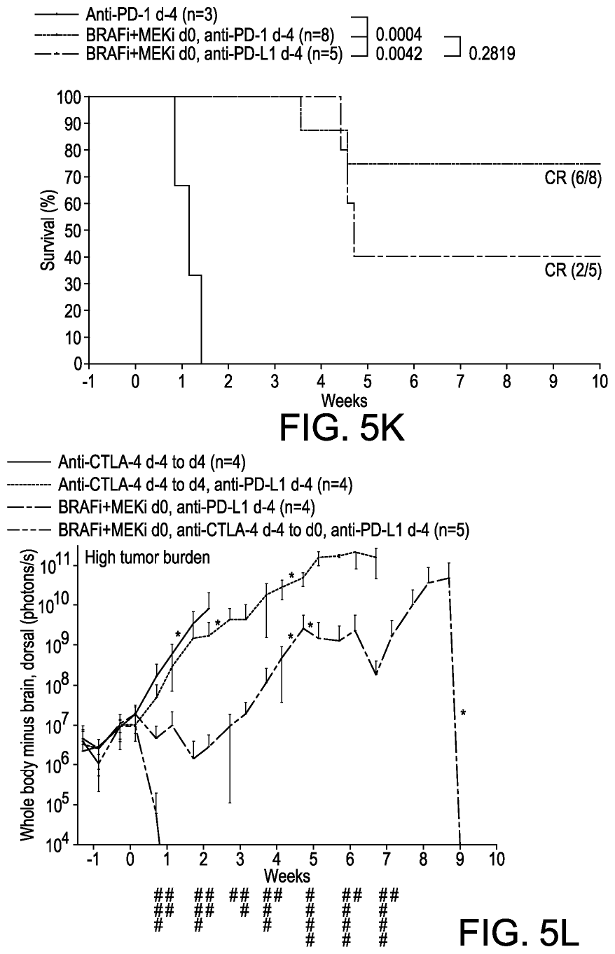

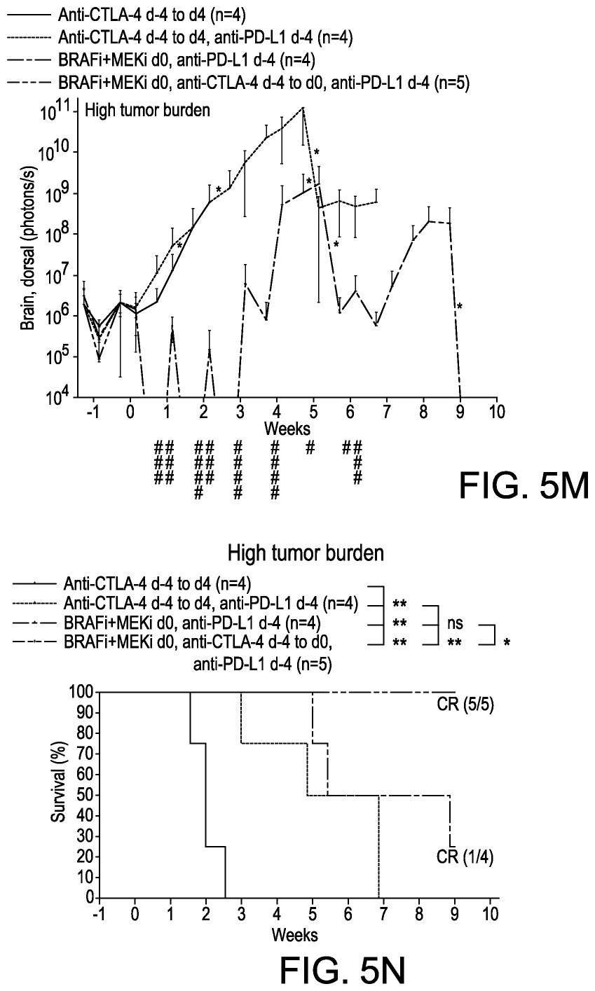

Since responses of MBM to therapies may be inferior, we tested sequencing-combinatorial regimens for their antitumor impacts across multiple organ sites. We engineered Braf^{V600E} (YUMM1.7ER) melanoma cells to express luciferase and injected YUMM1.7ER-Luc cells into the left ventricle. By in vivo bioluminescence imaging (BLI), dissemination of Braf^{V600E} melanoma cells resulted in tumor growth in 100% of mice in both intracranial and extracranial sites 8d after intracardiac (IC) injection (FIG. 5A). In vivo BLI signals (ventral or dorsal extracranial and intracranial) grew from a nadir on d3 to d4 after IC injection and surpassed an average of 1e9 by ˜d21 after IC injection (FIGS. 5B to 5E and 10A). Median survival of untreated mice (defined in FIG. 5B) was 3.5 weeks after IC injection (n=19) (FIG. 5F). When the dorsal intracranial BLI signals averaged 1e6-1e7 on day 8 (when 100% of mice harbored MBM) after IC injection, we designated this time point of initiating MEKi-containing regimens as do (FIG. 5A). At necropsy when untreated mice were moribund, ex vivo BLI revealed tumor burden in the lung, adrenal glands, ovaries, pancreas, and brain in 100% of mice (FIG. 5B) as well as in the kidney (6/13 or 46%), heart (9/13 or 69%), and liver (6/13 or 46%) (FIG. 10B).

To evaluate impacts on multi-organ metastatic growth, we compared the following regimens: (i) anti-PD-L1 d-4 or d0; (ii) MEKi d0; (iii) MEKi d0 plus anti-PD-L1 d-4, d0, or d4 (FIG. 5A). Although anti-PD-L1 d0 elicited no discernable impact on extracranial or intracranial metastatic growth (FIGS. 5C, 5D, 10C and 10D), anti-PD-L1 d-4 suppressed MBM (FIGS. 5D and 10D) and extended survival (FIG. 5F). MEKi d0 clearly reduced extracranial metastatic tumor burden in surviving mice past 4 weeks (FIGS. 5B, 5C, 5E, and 10C) but only delayed MBM by ˜1.5 weeks (FIGS. 5B, 5D, 5E, S4C and S4D), which limited the survival benefit of MEKi monotherapy (FIG. 5F) (Davies et al., 2017; Seifert et al., 2016). Comparing among the group (iii) regimens, we observed that the regimen of MEKi d0, anti-PD-L1 d-4 was superior in controlling both extracranial and intracranial tumor burdens (FIGS. 5C, 5D, S4C and 10D). Survival of mice on the regimen of MEKi d0, anti-PD-L1 d-4 was superior to that in every other group, except the regimen of MEKi d0, anti-PD-L1 d0 (FIG. 5E). In contrast, survival of mice on the regimen of MEKi d0, anti-PD-L1 do was not significantly superior to that on any other group except for the no treatment group and the anti-PD-L1 monotherapy groups (FIG. 5F). Thus, in mice with metastatic Braf^{V600E} melanoma, two doses of anti-PD-L1 prior to its combination with MEKi overcame innate anti-PD-L1 resistance and delayed acquired MEKi resistance in MBM.

We further evaluated the two most efficacious group iii regimens, namely MEKi d0, anti-PD-L1 d-4 and MEKi d0, anti-PD-L1 d0, by substituting MEKi monotherapy with BRAFi+MEKi combo therapy. As expected, both of these regimens were superior in efficacy to BRAFi+MEKi (FIG. 5G). Importantly, the regimen of BRAFi+MEKi d0, anti-PD-L1 d-4 (median survival, not reached at 10 weeks) trended (p=0.0543) toward greater survival benefit compared to the regimen of BRAFi+MEKi d0, anti-PD-L1 d0 (median survival of 4 weeks) (FIG. 5G). We devised a protocol to follow surviving mice longer-term, up to about ˜34 weeks (FIG. 10E). For mice treated on the regimen of BRAFi+MEKi d0, anti-PD-L1 d-4, the median survival was reached at ˜13.5 weeks (FIG. 10F), which tripled mice survival on the regimen of BRAFi+MEKi d0, anti-PD-L1 d0. Moreover, two long-term survivors displayed confirmed or true CRs. We then challenged the two most efficacious regimens with greater metastatic tumor burden (i.e., high tumor burden) by delaying the start of BRAFi+MEKi treatment by two days post-IC injection (FIG. 5H). Although the survival benefits of both regimens were decreased, run-in with anti-PD-L1 prior to combination with BRAFi+MEKi yielded superior survival benefit compared with simultaneously initiating all three therapeutic agents (p=0.0006) (FIG. 5H). In the context of high tumor burden, we also evaluated the most efficacious group (BRAFi+MEKi d0, anti-PD-L1 d-4) by substituting anti-PD-L1 with anti-PD-1. Similar to the observations from a subcutaneous tumor model (FIG. 2H), the antitumor (FIGS. 5I and 5J) and pro-survival (FIG. 5K) activity of the regimen BRAFi+MEKi d0, anti-PD-1 d-4 was greater than that of anti-PD-1 d-4. Furthermore, since anti-PD-1 plus anti-CTLA-4 improve anti-melanoma and anti-MBM efficacy clinically (Tawbi et al., 2018; Wolchok et al., 2017), we tested whether anti-CTLA-4 (2 doses from d-4 to do) in the high tumor burden model would further the priming action of anti-PD-L1 (FIGS. 5L to 5N). Indeed, priming with combined ICT improved the antitumor activity (FIGS. 5L and 5M) and survival benefit (FIG. 5N) of the sequential-combinatorial regimen.

### Anti-PD-1/L1 Priming of MAPKi Responses Via T-Cell Clonal Expansion

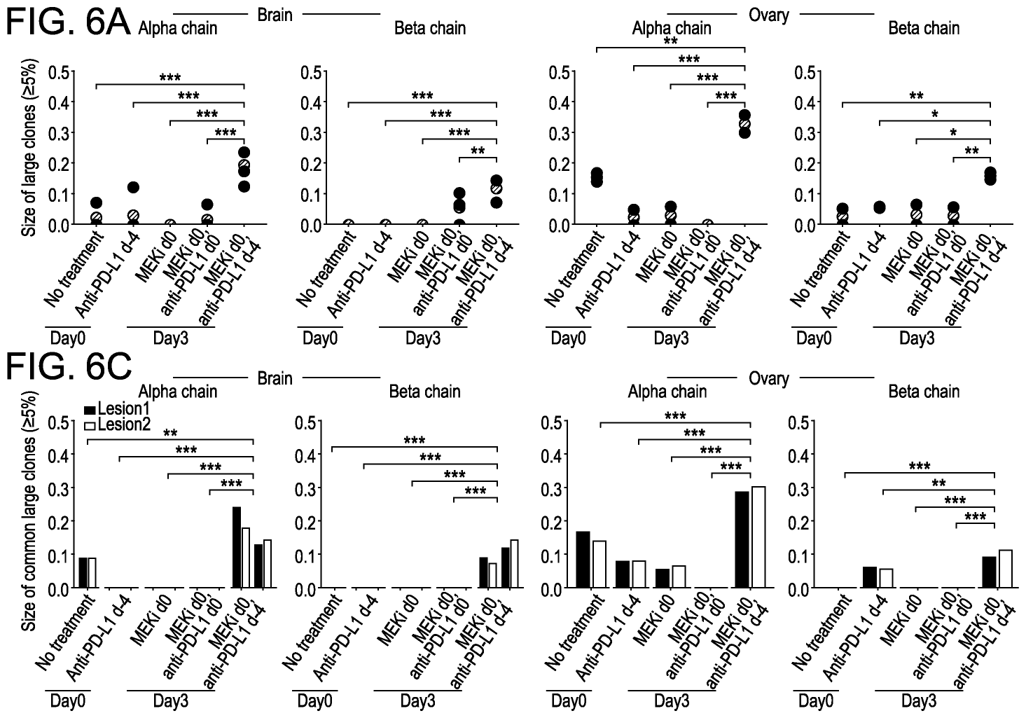

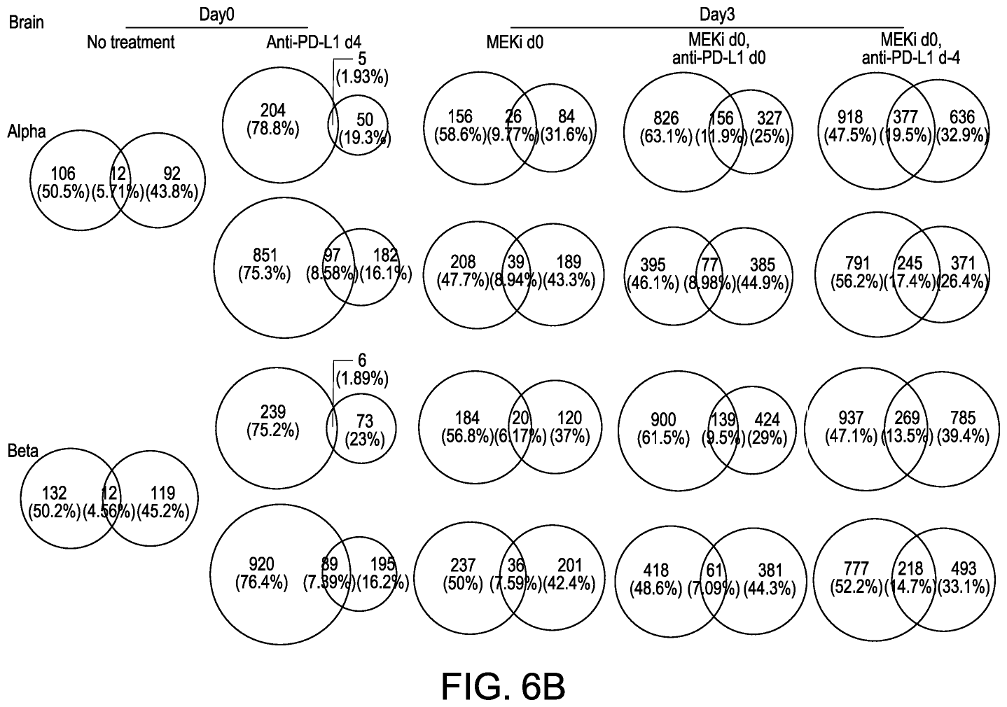

We tracked the intratumoral (ovarian and brain tumors) T-cell clonotypes by TCR-seq of five regimens at do and d3 (FIG. 5A). We excluded from this analysis two regimens (anti-PD-L1 d0; MEKi d0, anti-PD-L1 d4), which respectively lacked superiority over the no treatment group and over the regimens of MEKi d0 or MEKi d0, anti-PD-L1 d0. We calculated the Gini or clonality and diversity indices and observed that T-cell clonality (based on a and b chains) was higher on d3 after treatment with MEKi d0, anti-PD-L1 d-4 than that in any other regimen in both tumor-involved brain and ovarian tissues (FIG. 10G). Consistently. TCR diversity was lower on d3 after treatment with MEKi d0, anti-PD-L1 d-4 especially compared to the d3 tissues on-treatment with MEKi d0 or MEKi d0, anti-PD-L1 do. We also analyzed the sizes of large (≥5%) TCR clones and found that the regimen of MEKi d0, anti-PD-L1 d-4 on d3 led to the highest accumulation of large TCR clones (on average 10-20% in tumor-involved brain or 15-35% in tumor-involved ovaries) (FIG. 6A). The pattern was consistent when we analyzed the sizes of large clones defined as ≥8% or of the largest, top 5 or top 10 clones (FIGS. 10H and 10I). Moreover, we calculated the fractions of overlapping TCR clones between two distinct regions of tumor-involved brain tissues in each animal (FIG. 6B). Increasing overlap may reflect greater tumor-specific T-cell expansion. In this regard, the fraction ranged from 4.56-5.71% on do in untreated mice; 1.89-8.58% on do in mice treated with anti-PD-L1 d-4; 6.17-9.77% on d3 in mice treated with MEKi d0; 7.09-11.90% on d3 in mice treated with MEKi d0, anti-PD-L1 d0; and, importantly, 13.50-19.50% on d3 in mice treated with MEKi d0, anti-PD-L1 d-4. The differences in this overlap between the most efficacious regimen (MEKi d0, anti-PD-L1 d-4) and MEKi d0 monotherapy were significant (p=0.045 and 0.017 for a and b chain, respectively; Student's t-test). Thus, the regimen of anti-PD-L1 lead-in followed by MEKi combination led to greater geographic convergence of TCR clones across distinct brain tumor lesions. Furthermore, within the overlapping TCR clones between two distinct tumor-involved lesions from brain or ovary tissues in each mouse, we calculated the sizes of common large (≥5%) TCR clones (FIG. 6C). For both a or b TCR chains and for both brain and ovary tissues, the average size was greatest from tumor-bearing mice treated with anti-PD-L1 lead-in followed by MEKi combination. In further analysis, we calculated the fractions of overlapping TCR clones between all tumor-involved brain and ovary tissues (FIG. 10J). The average of fractions for the a and b TCR chains from the regimen of MEKi d0, anti-PD-L1 d-4 trended higher versus any other group. Hence, the regimen consisting of two doses of anti-PD-L1 followed by MEKi combination elicited the most robust T-cell clonal expansion and clonotypic convergence between distinct tumor-involved regions of each organ and across intracranial and extracranial organs.

### DISCUSSION

Combination therapies are typically initiated together. Whether sequencing therapies instead of or in addition to combining therapies would augment antitumor efficacy remains under explored. Here, we found that sequencing of just two doses of anti-PD-1/L1 (two doses of anti-CTLA-4, without further dosing) prior to MAPKi combination maximizes antitumor immunity and efficacy.

Post hoc analyses of clinical data performed here and published previously (Dummer et al., 2017) in patients (with BRAF^{V600MUT} or NRAS^{MUT} melanoma) treated with MAPKi found increased benefit from MAPKi in those who have been treated with ICT immediately prior to MAPKi. That antitumor immunity may be a critical element elicited by and modulating the efficacy of MAPK-targeted therapy was suggested by our prior studies of patients with BRAF^{V600MUT} melanoma whose acquired MAPKi-resistant melanoma display signs of immune evasion (Hugo et al., 2015; Hugo et al., 2016; Song et al., 2017). Based on the current study, superior antitumor activity of the regimen consisting of anti-PD-1/L1 lead-in followed by MAPKi combination was positively linked to multiple cell types, including M1-like TAMs, CD4⁺ Th1, and CD8⁺ T cells. Thus, strategies to target M2-like TAMs and to improve clonal expansion and persistence of tumor-specific CD8⁺ cytotoxic T cells may further improve the proposed sequential-combinatorial strategy.

The finding here that anti-PD-1/L1 lead-in followed by MAPKi combination can improve anti-MBM activity (and thereby survival) in mice has immediate clinical implications. This sequential-combinatorial strategy may present an alternative strategy to combination ICT with anti-PD-1+anti-CTLA-4, which improves anti-MBM responses elicited by anti-PD-1 monotherapy (Tawbi et al., 2018). Melanoma has a high propensity for CNS metastasis (40-80%), which is associated with poor overall survival (median 4-5 months) (Davies et al., 2011). CNS is often the initial site of acquired resistance (which often persists in isolation without extracranial disease progression) in patients on MAPKi (Frenard et al., 2016; Long et al., 2016). The experimental metastatic model established here recapitulated the propensity of MBM to escape from MAPKi therapy. Importantly, sequencing of anti-PD-1/L1 therapy prior to MAPKi addition resulted in the most durable anti-MBM activity. This benefit may be extended by priming with anti-PD-1/L1 plus anti-CTLA-4 (with a limited duration of anti-CTLA-4 dosing to avoid added toxicities).

Data presented here support the conclusions that anti-PD-1/L1 therapy primes a more durable MAPKi response and that MAPKi combination helps to overcome innate anti-PD-1/L1 resistance. These findings prompted the design and initiation of a single-site clinical trial (NCT04375527) testing the activity of binimetinib plus nivolumab in patients with BRAF^{V600WT} melanoma that display innate resistance to ICT. Findings here should concentrate a larger effort to evaluate prospectively the activity of anti-PD-1/L1 agents combined with BRAFi+MEKi or MEKi (in BRAF^{V600MUT} or BRAF^{V600WT} melanoma, respectively) after distinct predefined periods of prior ICT exposure, with or without objective evidence of innate resistance to ICT. Moreover, the benefit of triplet therapy with anti-PD-1/L1+BRAFi+MEKi may be clinically meaningful for anti-PD-1/L1-experienced (vs. naïve) patients despite objective evidence of innate ICT resistance and for patients with symptomatic or non-symptomatic MBM.

While the animal model studies do not address the impacts of prolonging anti-PD-1/L1 lead-in before MAPKi combination, these in vivo models collectively serve as an important platform to compare and mechanistically dissect alternative sequential-combinatorial regimens, including those inclusive of additional targeted agents.

### REFERENCES

Ackerman, A., et al. (2014). Cancer 120, 1695-1701.
Algazi, A. P., et al. (2020). Nat Med 26, 1564-1568.
Ascierto, P. A., et al. (2019). Nat Med 25, 941-946.
Butler, A., et al. (2018). Nat Biotechnol 36, 411-420.
Chen, H., et al. (2016). PLoS Comput Biol 12, e1005112.
Davies, M. A., et al. (2011). Cancer 117, 1687-1696.
Davies, M. A., et al. (2017). Lancet Oncol 18, 863-873.
Dummer, R., et al. (2017). Lancet Oncol 18, 435-445.
Flaherty, K. T., et al. (2012). N Engl J Med.
Frenard, C., et al. (2016). J Neurooncol 126, 355-360.
Ghebremedhin, A., et al. (2020). bioRxiv.
Gutzmer, R., et al. (2020). Lancet 395, 1835-1844.
Hong, A., et al. (2018). Cancer Discov 8, 74-93.
Hong, A., et al. (2021). Cancer Discov 11, 714-735.
Hugo, W., et al. (2015). Cell 162, 1271-1285.
Hugo, W., et al. (2016). Cell 165, 35-44.
Jaynes, J. M., et al. (2020). Sci Transl Med 12.
Johnson, D. B., et al. (2017). J Immunother 40, 31-35.
Kotecha, N., et al. (2010). Curr Protoc Cytom Chapter 10, Unit10 17.
Long, G. V., et al. (2016). Lancet Oncol 17, 1743-1754.
Mason, R., et al. (2020). Pigment Cell Melanoma Res 33, 358-365.
Nazarov, V. I., et al. (2015). BMC Bioinformatics 16, 175.
Reijers, I. L. M., et al. (2020). Pigment Cell Melanoma Res 33, 498-506.
Ribas, A., et al. (2016). JAMA 315, 1600-1609.
Ribas, A., et al. (2019). Nat Med 25, 936-940.
Seifert, H., et al. (2016). Pigment Cell Melanoma Res 29, 92-100.
Siddiqui, I., et al. (2019). Immunity 50, 195-211 e110.
Simeone, E., et al. (2017). Oncoimmunology 6, e1283462.
Song, C., et al. (2017). Cancer Discov 7, 1248-1265.
Tawbi, H. A., et al. (2018). N Engl J Med 379, 722-730.
Tetu, P., et al. (2018). Eur J Cancer 93, 147-149.
Wang, J., et al. (2017). Pigment Cell Melanoma Res 30, 428-435.
Wolchok, J. D., et al. (2017). N Engl J Med 377, 1345-1356.

TABLE 1
For S1320 trial, associations between baseline patient characteristics and
prior ICT, related to FIG. 1. Median (range) or N (%) reported.

| | No prior exposure | | Prior checkpoint | | P- |
|---|---|---|---|---|---|
| Factor | (N = 145) | | therapy (N = 61) | | value |
| Randomized arm | | | | | |
| Continuous Dosing | 74 | (51) | 31 | (51) | 1 |
| Intermittent Dosing | 71 | (49) | 30 | (49) | |
| Age at randomization (years) | 62 | (52, 69) | 58 | (47, 67) | 0.16 |
| Age at randomization (years) | | | | | |
| Younger than 45 | 24 | (17) | 15 | (25) | 0.18 |
| 45 and older | 121 | (83) | 46 | (75) | |
| Gender | | | | | |
| Female | 51 | (35) | 23 | (38) | 0.75 |
| Male | 94 | (65) | 38 | (62) | |
| Race | | | | | |
| White | 142 | (98) | 59 | (97) | 0.6 |
| Asian and white | 1 | (1) | 0 | (0) | |
| Native American | 0 | (0) | 1 | (2) | |
| Unknown race | 2 | (1) | 1 | (2) | |
| Ethnicity | | | | | |
| Hispanic | 4 | (3) | 2 | (3) | 1 |
| Not hispanic | 141 | (97) | 59 | (97) | |
| PS | | | | | |
| 0 | 82 | (57) | 35 | (58) | 0.95 |
| 1 | 60 | (42) | 24 | (40) | |
| 2 | 2 | (1) | 1 | (2) | |
| LDH at randomization | | | | | |
| Elevated LDH | 53 | (37) | 24 | (39) | 0.75 |
| Normal LDH | 92 | (63) | 37 | (61) | |
| Primary | | | | | |
| Cutaneous primary | 112 | (78) | 52 | (88) | 0.12 |
| Unknown primary | 32 | (22) | 7 | (12) | |
| Stage | | | | | |
| M0 | 14 | (10) | 11 | (18) | 0.41 |
| M1A | 25 | (17) | 11 | (18) | |
| M1B | 32 | (22) | 12 | (20) | |
| M1C | 74 | (51) | 27 | (44) | |
| Response | | | | | |
| CR | 13 | (9) | 9 | (15) | 0.43 |
| Unconfirmed CR | 3 | (2) | 0 | (0) | |
| PR | 76 | (54) | 38 | (64) | |
| Unconfirmed PR | 22 | (16) | 6 | (10) | |
| Stable | 22 | (16) | 5 | (8) | |
| Increasing disease | 2 | (1) | 0 | (0) | |
| Inadequate assessment | 3 | (2) | 1 | (2) | |

Tables 2A & 2B. Multivariable Cox regression models for PFS (A) and OS (B), Related to FIG. 1.

TABLE 2A

| Covariate | HR | 80% Cl | P-value |
|---|---|---|---|
| Prior checkpoint therapy | 0.6 | (0.47, 0.77) | 0.009 |
| (ref = No prior exposure) | | | |
| Intermittent Dosing | 1.57 | (1.27, 1.96) | 0.0073 |
| (ref = Continuous Dosing) | | | |
| Age at randomization (years) | 0.99 | (0.98, 1) | 0.17 |
| Male (ref = Female) | 0.93 | (0.73, 1.18) | 0.69 |
| PS 1 (ref = PS 0) | 1.3 | (1.04, 1.62) | 0.13 |
| PS 2 (ref = PS 0) | 3.2 | (1.23, 8.34) | 0.12 |
| Normal LDH | 0.63 | (0.5, 0.79) | 0.01 |
| (ref = Elevated LDH) | | | |
| Unknown primary | 0.71 | (0.53, 0.95) | 0.13 |
| (ref = Cutaneous primary) | | | |
| M1A (ref = M0) | 1.24 | (0.79, 1.95) | 0.53 |
| M1B (ref = M0) | 1.6 | (1.04, 2.46) | 0.16 |
| M1C (ref = M0) | 1.9 | (1.27, 2.82) | 0.039 |

TABLE 2B

| Covariate | HR | 80% Cl | P-value |
|---|---|---|---|
| Prior checkpoint therapy | 0.86 | (0.63, 1.17) | 0.52 |
| (ref = No prior exposure) | | | |
| Intermittent Dosing | 1.13 | (0.85, 1.49) | 0.58 |
| (ref = Continuous Dosing) | | | |
| Age at randomization (years) | 1 | (0.99, 1.01) | 0.81 |
| Male (ref = Female) | 0.63 | (0.47, 0.85) | 0.051 |
| PS 1 (ref = PS 0) | 2.16 | (1.64, 2.85) | <0.001 |
| PS 2 (ref = PS 0) | 12.71 | (4.48, 36.06) | 0.0018 |
| Normal LDH | 0.54 | (0.41, 0.73) | 0.0069 |
| (ref = Elevated LDH) | | | |
| Unknown primary | 0.65 | (0.44, 0.95) | 0.15 |
| (ref = Cutaneous primary) | | | |
| M1A (ref = M0) | 1.67 | (0.79, 3.5) | 0.38 |
| M18 (ref = M0) | 3.01 | (1.48, 6.13) | 0.047 |
| M1C (ref = M0) | 3.61 | (1.84, 7.09) | 0.015 |

TABLE 3
Expansion indices as well as top 5 and 10 clone sizes of CD8+ TC, Ki-67hi CD8+
T and T_{REG} cells in each treatment regimen group and time point, Related to FIG. 3. See FIG. 2A.

| Expansion index | | | Size of top5 clones | | | Size of top10 clones | | | |
|---|---|---|---|---|---|---|---|---|---|
| CD8⁺ T_{C} | Ki-67^{hi} CD8⁺ | T_{REG} | CD8⁺ T_{C} | Ki-67^{hi} CD8⁺ | T_{REG} | CD8⁺ T_{C} | Ki-67^{hi} CD8⁺ | T_{REG} | |
| 0.0630 | 0.0000 | 0.0086 | 0.0465 | 0.0159 | 0.0171 | 0.0734 | 0.0159 | 0.0282 | Day 7 |
| 0.1796 | 0.0204 | 0.0000 | 0.1478 | 0.1063 | 0.1113 | 0.1777 | 0.1096 | 0.1213 | |
| 0.1852 | 0.0000 | 0.0074 | 0.1022 | 0.0424 | 0.0849 | 0.1283 | 0.0424 | 0.0945 | Day 10 |
| 0.0193 | 0.0000 | 0.0000 | 0.1129 | 0.0000 | 0.0484 | 0.1532 | 0.0000 | 0.0887 | |
| 0.1392 | 0.0817 | 0.0000 | 0.2160 | 0.0488 | 0.0418 | 0.3031 | 0.0488 | 0.0592 | |
| 0.1416 | 0.0817 | 0.0000 | 0.1203 | 0.0253 | 0.0190 | 0.1582 | 0.0253 | 0.0190 | |
| 0.0067 | 0.0000 | 0.0067 | 0.0516 | 0.0299 | 0.0387 | 0.0616 | 0.0299 | 0.0464 | Day 14 |
| 0.1175 | 0.0000 | 0.0066 | 0.0235 | 0.0063 | 0.0093 | 0.0334 | 0.0066 | 0.0136 | |
| 0.2392 | 0.0000 | 0.0334 | 0.1800 | 0.0284 | 0.1411 | 0.2141 | 0.0284 | 0.1492 | |
| 0.2210 | 0.0245 | 0.0000 | 0.1018 | 0.0583 | 0.0317 | 0.1372 | 0.0590 | 0.0398 | |

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method of enhancing efficacy of mitogen-activated protein kinase inhibitor (MAPKi) therapy or immune checkpoint therapy (ICT), the method comprising:
(a) pretreating a subject in need of MAPKi therapy by administering one to two doses of ICT to the subject;
(b) subsequent to the pretreating of (a), administering to the subject a combination of MAPKi and ICT.

2. The method of claim 1, wherein the ICT comprises an anti-PD-1 or anti-PD-L1 agent.

3. The method of claim 2, wherein the ICT further comprises an anti-CTLA-4 agent.

4. The method of claim 1, wherein the subject has a BRAF V600 mutant cancer and the MAPKi is a BRAF inhibitor (BRAFi) or a BRAF plus a MEK inhibitor (MEKi).

5. The method of claim 1, wherein the subject has a NRAS, KRAS, and/or NF1 mutant cancer and the MAPKi is a MEKi.

6. The method of claim 1, wherein the pretreating of (a) consists of administering one to two doses over one to eight weeks.

7. The method of claim 1, wherein the administering of (b) begins two to eight weeks following the initiation of the pretreating of (a).

8. The method of claim 1, wherein the pretreating of (a) further comprises administering one to two additional doses of ICT.

9. The method of claim 1, wherein the pretreating of (a) further comprises administering a modulator of regulatory T cells and/or M2-like TAMs.

10. The method of claim 9, wherein the modulator of regulatory T cells is a CD25-depleting antibody.

11. The method of claim 9, wherein the modulator of M2-like TAMs is a peptide agonist of CD206.

12. The method of claim 8, wherein the pretreating of (a) begins two to eight weeks prior to the administering of (b).

13. A method of suppressing melanoma metastasis in a subject, the method comprising:
(a) pretreating a subject in need of treatment for melanoma metastasis by administering one to one to two doses of ICT to the subject;
(b) subsequent to the pretreating of (a), administering to the subject a combination of MAPKi and ICT.

14. The method of claim 13, wherein the metastasis is a brain metastasis.

15. A method of enhancing the inhibition of M2-like tumor associated macrophages (TAMs) in a subject, the method comprising:
(a) pretreating a subject in need of inhibition of M2-like TAMs by administering one to one to two doses of ICT plus a pharmacologic M2-like TAM inhibitor to the subject;
(b) subsequent to the pretreating of (a), administering to the subject a combination of MAPKi and ICT.

16. The method of claim 15, wherein the inhibiting of M2-like TAMs effects a selective upregulation of pro-inflammatory or M1-like TAMs in the subject.
